(19) United States
(12) Patent Application Publication
(10) Pub. No.: US 2026/0306670 A1
(43) Pub. Date: Oct. 8, 2026

(54) BASE EDITING APPROACHES TO TREAT ABCA4-ASSOCIATED STARGARDT DISEASE

(71) Applicant: Massachusetts Eye & Ear Infirmary, Boston, MA (US)

(72) Inventors: Caitlin Collin, Boston, MA (US); Qin Liu, Boston, MA (US)

(21) Appl. No.: 19/482,510

(22) PCT Filed: May 10, 2024
(86) PCT No.: PCT/US2024/028886

§ 371 Date: Nov. 7, 2025

Related U.S. Application Data
(60) Provisional application No. 63/465,758, filed on May 11, 2023.

(51) Int. Cl. A61K 48/00; A61K 38/17; C12N 9/22; C12N 9/78; C12N 15/11; C12N 15/86; C12N 15/90

(52) U.S. Cl. CPC A61K 48/005; A61K 38/1709; A61K 48/0075; C12N 9/226; C12N 9/78; C12N 15/111; C12N 15/86; C12N 15/907; C07K 2319/09; C12N 2310/20; C12N 2750/14143; C12Y 305/04001; C12Y 305/04002

(57) ABSTRACT

Described herein are base editors (BEs)/single guide RNAs (sgRNAs) and their use in methods of treating recessive retinal disease caused by mutations in ABCA4.

### CLAIM OF PRIORITY

This application claims the benefit of U.S. Provisional Patent Application Ser. No. 63/465,758, filed on May 11, 2023. The entire contents of the foregoing are hereby incorporated by reference.

### TECHNICAL FIELD

Described herein are base editors/guide RNAs (BEs/gRNAs) complexes and their use in methods of treating recessive retinal disease caused by mutations in ABCA4.

### BACKGROUND

Stargardt disease (STGD) is the most common cause of inherited macular degeneration beginning in early childhood or adolescence. Autosomal recessive Stargardt disease 1 (STGD1) has an estimated prevalence of 1:8,000-10,000, and is associated with variants in the ATP-binding cassette transporter type A4 (ABCA4) gene [1]. ABCA4 encodes a large multidomain transmembrane protein which is largely retina specific, and localizes in the outer segment disk edges of rod photoreceptors [2]. ABCA4 functions as an important transporter of vitamin A derivatives, which facilitates removal of retinoid products from the disc membranes, and prevents the accumulation of toxic lipofuscins in the RPE [3]. These fatty deposits increase around the macula and impair central vision, eventually leading to photoreceptor death and further vision loss in people that lack the ABCA4 protein.

There are currently no therapies for Stargardt disease, despite promising advances in gene augmentation therapy, especially for Inherited Retinal Degenerations (IRDs) [4]. Notably, the first gene-based approach to be approved for use in patients is an AAV mediated augmentation of RPE65 as a therapy for Leber congenital amaurosis [5]. However, the large size of the ABCA4 coding sequence, at 6.8 kb, coding for 2273 amino acids, far exceeds the 4.7 kb packaging capacity of conventional AAV delivery vectors [6]. Alternative delivery methods are being explored, via dual AAVs [7], lentivirus [8], and nanoparticles [9], but still face challenges around safety and efficiency of delivery to the target cells, as well as long-term maintenance of appropriate levels of gene expression. Furthermore, these approaches do not correct the fundamental defect underlying the disease state. The present disclosure overcomes these translational barriers by using a base editing approach for ABCA4-associated diseases. Base editing systems are capable of maintaining the edited gene under its endogenous regulatory elements by directly altering the genomic ABCA4 loci, thereby avoiding ectopic expression and abnormal gene production that may occur with conventional viral-mediated gene augmentation therapies.

### SUMMARY

Provided herein are methods, complexes, vectors, compositions, and kits for modifying a polynucleotide (e.g., genomic DNA) using a complex, known as “Base editor (BE)”, comprising a fusion protein comprising a deaminase (e.g., cytidine deaminase or adenosine deaminase) and a nucleic acid programmable DNA binding protein (“napDNAbp”), and a guide RNA (gRNA), to treat ABCA4 associated Stargardt disease.

In some embodiments, the present disclosure provides methods of editing the ABCA4 gene locus using a base editor (BE) that comprises a napDNAbp domain (e.g., Cas9 nuclease, or an ortholog, homolog, or variant thereof), and one or more deaminase domains (e.g., a cytidine or adenosine deaminase), which when complexed with a suitable guide RNA, are effective in treating conditions associated with a mutation in the ABCA4 gene by installing one or more edits (e.g., replacement of one or more nucleobases) in the ABCA4 genomic locus.

In some embodiments, the one or more nucleobase edits installed in the ABCA4 gene locus result in a modified ABCA4 gene product having increased stability, activity, and/or production relative to an unedited mutant ABCA4 gene product. In some embodiments, deaminating an adenosine nucleobase in the ABCA4 gene ameliorates one or more symptoms of Stargardt disease, such as partially or completely restoring visual loss. In other embodiments, deaminating a cytosine nucleobase in the ABCA4 gene ameliorates one or more symptoms of Stargardt disease, such as partially or completely restoring visual loss.

Accordingly, aspects of the disclosure provide methods and compositions for deaminating an adenine nucleobase (A) in an ABCA4 gene using a complex of an adenine base editor (ABE) bound to a guide RNA (gRNA), wherein the gRNA comprises a guide sequence that is complementary to a target nucleic acid sequence in the ABCA4 gene.

Other aspects of the disclosure provide methods and compositions for deaminating a cytosine nucleobase (C) in an ABCA4 gene using a complex of a cytidine base editor (CBE) bound to a guide RNA (gRNA), wherein the gRNA comprises a guide sequence that is complementary to a target nucleic acid sequence in the ABCA4 gene. In some embodiments, the disclosure provides complexes comprising an adenine base editor (ABE) and a guide RNA (gRNA).

In some embodiments, the gRNA directs the base editor within 1, 2, 3, 4, 5, 6, 7, 8, 9, 10, 11, 12, 13, 14, 15, 16, 17, 18, 19, or 20 base pairs of a point mutation within the ABCA4 gene. In some embodiments, the guide RNA comprises a nucleic acid sequence selected from the group of guide sequences in Table 2, e.g., comprising one of SEQ ID NOs:6 to 37. Exemplary guide sequences to target ABCA4 may comprise any of the guide sequences set forth herein, e.g., as SEQ ID NOs: 6-37. In some embodiments, the gRNA comprises a protospacer sequence that comprises any of the nucleic acid sequences as set forth herein, e.g., in SEQ ID NOs: 6-37. The protospacer sequence has the same nucleotide sequence as the spacer sequence of the guide RNA, except the spacer sequence comprises ribonucleotides (e.g., uracils), and the protospacer sequence comprises deoxyribonucleotides (e.g., thymines).

Further provided are BE/gRNA complexes, and compositions thereof, for use in the preparation of a medicament and for use in treating a subject having or a subject suspected of having ABCA4 associated disease or Stargardt disease.

The present disclosure further provides vectors comprising one or more polynucleotides and vectors encoding a base editor and a guide RNA, or both. In some embodiments, the vectors are recombinant adeno-associated virus (rAAV) vectors. In some embodiments, the vectors are plasmids. rAAV vectors are an efficient and widely used delivery agent that remains the only FDA-approved in vivo gene therapy vector. Further provided herein are kits comprising one or more of these vectors (e.g., rAAV vectors).

In some embodiments involving an adenosine base editor, the step of contacting a target nucleic acid with a base editor results in the deamination of a adenosine nucleobase in the ABCA4 gene, which results in a T:A base pair in the ABCA4 gene being converted to a C:G base pair in the ABCA4 gene (a transition substitution of an “A” to a “G”). In some embodiments, the step of contacting results in deamination of the adenosine nucleobase in the ABCA4 gene that restores (partially or completely) functional ABCA4 protein. In some embodiments, deaminating the adenosine nucleobase in the ABCA4 gene corrects a missense mutation or a nonsense mutation that restores functional ABCA4 protein.

In other embodiments involving a cytidine base editor, the step of contacting a target nucleic acid with a base editor results in the deamination of a cytidine nucleobase in the ABCA4 gene, which results in a C:G base pair in the ABCA4 gene being substituted with a T:A base pair in the ABCA4 gene (a transition substitution of a “C” with a “T”). In some embodiments, the step of contacting results in the deamination of a cytidine nucleobase in the ABCA4 gene that restores (partially or completely) functional ABCA4 protein. In some embodiments, deaminating the cytidine nucleobase in the ABCA4 gene corrects a missense mutation or a nonsense mutation that restores functional ABCA4 protein.

The complexes, vectors, rAAV particles, and compositions of the disclosure may be formulated for delivery to the eye (e.g., the retina) of a subject. In some embodiments, the subject is diagnosed with, or is suffering from, a disease associated with a deficiency in functional ABCA4 protein expression. In some embodiments, the subject is diagnosed with, or is suffering from, Stargardt disease. Accordingly, provided herein are methods of treatment comprising the administration of one or more of the disclosed complexes, vectors, rAAV particles, and compositions to a subject, e.g., a human subject suffering from ABCA4 associated Stargardt disease.

Thus, provided herein are compositions comprising (i) a single guide RNA (sgRNA) comprising a Protospacer Sequence, e.g., as described in Table 2 or 3, e.g., comprising one of SEQ ID NOs:6 to 37, and optionally a corresponding base editor (BE), optionally an adenine base editor (ABE) or cytosine base editor (CBE), as indicated in Table 2 or 3). In some embodiments, the compositions comprise ribonucleoprotein complexes comprising the sgRNA/BE. In some embodiments, the compositions comprise nucleic acids encoding the sgRNA/BE, or the sgRNA and nucleic acids encoding the BE, respectively.

In some embodiments, the nucleic acids comprising mRNA or cDNA encoding the BE proteins further comprise a nuclear localization signal, optionally a C-terminal nuclear localization signal and/or an N-terminal nuclear localization signal; and/or wherein the sequences encoding the BE comprise a polyadenylation signal.

In some embodiments, the compositions comprise the nucleic acids comprise mRNA or cDNA encoding the BE proteins, optionally wherein the nucleic acids are naked or in an expression vector comprising a regulatory sequence such as a promoter that drives expression of the protein. In some embodiments, the expression vectors are viral delivery vectors, optionally an AAV, preferably wherein the AAV further comprises a first and a second inverted terminal repeat sequence (ITR). In some embodiments, the viral delivery vector comprises a promoter that drives expression of the BE, preferably a CMV, EFS, or hGRK1 promoter. In some embodiments, the AAV vector is formulated to be delivered to a retina of a subject by injection, such as by subretinal injection.

Also provided are the compositions described herein for use in therapy, for use in preparation of a medicament, or for use in a method of treating a subject who has a condition associated with a mutation in an ABCA4 gene, e.g., wherein the condition is Stargardt disease.

Further, provided herein are methods for altering a genome of a cell, comprising contacting the cell with a composition as described herein. In some embodiments, the cell is from a subject who has Stargardt disease. In some embodiments, the cell is a retinal cell or a photoreceptor cell. In some embodiments, the photoreceptor cell is a cone photoreceptor cell or a cone cell, a rod photoreceptor cell or a rod cell or a macular cone photoreceptor cell.

Additionally, provided herein are methods for altering a cell. The methods comprise contacting the cell with a recombinant viral particle comprising: a nucleotide sequence encoding an sgRNA comprising a Protospacer Sequence as described in Table 2, e.g., comprising one of SEQ ID NOs:6 to 37, and a corresponding base editor (BE), optionally an adenine base editor (ABE) or cytosine base editor (CBE), as indicated in Table 2 or 3), or wherein said viral particle is capable of delivery to a non-dividing cell, and wherein said contacting results in correction of a pathogenic mutation in an ABCA4 allele in the cell. In some embodiments, the viral particle is an adeno-associated virus (AAV) viral particle.

Also provided are methods for treating a subject who has a condition associated with a mutation in an ABCA4 allele. The methods comprise administering to the subject a therapeutically effective amount of a composition as described herein. In some embodiments, the subject has Stargardt disease. In some embodiments, the cell is a retinal cell or a photoreceptor cell. In some embodiments, the composition is delivered to a retina of a subject by injection, such as by subretinal injection. In some embodiments, the photoreceptor cell is a cone photoreceptor cell or a cone cell, a rod photoreceptor cell or a rod cell or a macular cone photoreceptor cell.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying Figures.

### DESCRIPTION OF DRAWINGS

FIGS. 1A-B Selection criteria for ABCA4 mutations. A. All reported mutations in ABCA4 were analyzed in The Human Gene Mutation Database (HGMD) (hgmd.cf.ac.uk). 38% were single nucleotide transition mutations that could theoretically be corrected by Adenine and Cytosine Base Editors (ABE and CBE). The eleven most common pathogenic mutations which cause Stargardt disease were selected (see Table 1) and the surrounding sequence analyzed to identify nearby PAM recognition sites of the napDNAbp(s). B. Schematic illustration of the identification of PAMs and the design of protospacer of guide RNAs for ABCA4 target sites. PAMs were identified which place the target base within the optimal editing window of the BEs. Consideration was given to other bases in the window that could be edited in a so-called ‘bystander’ effect.

FIGS. 2A-C. Experimental design and generation of mutant ABCA4 transgene lines in HEK293T cells. A. A synthetic gene comprising candidate ABCA4 mutations listed in Table 1, flanked by universal priming sequences. DNMT1 is included as a positive control site. Each target site contains around 51 bp of ABCA4 sequence flanking the target mutation. Restriction sites are included at each end to facilitate cloning into the multiple cloning site of a donor vector. B. The pAAVS1-EF1a-puro-DNR plasmid contains 550 bp left and right AAVS1 homologous sequences, a constitutive EF1a promoter, and a Puromycin selection cassette under the PGK promoter. Targeted insertion of the transgene into the human AAVS1 site occurs when co-transfected with pCas-Guide-AAVS1. C. Assessment of editing efficiency in HEK293T cell by transient co-transfection. The transgene donor vector was co-transfected with BE plasmids, and corresponding gRNA plasmids in a 1:1:1 molar ratio via Lipofectamine 3000 into HEK293T cells. 48 hours after transfection, cells were harvested, and the transgene was specifically amplified by PCR with universal primers. Editing was evaluated by Sanger sequencing, followed by EditR deconvolution, and amplicon Deep sequencing and Crispresso2 analysis.

FIGS. 3A-I. Base editing efficiencies at individual ABCA4 mutant sites.

A. ABE on Target 1—c.5882G>A. Highest editing (up to 40%) was observed at this site with sgRNA-mut1-PS2+Sp (NGG) ABE8e, which places the target nucleotide at position 3 of the protospacer. Bystander editing at position 4 results in a synonymous codon change. Deep sequencing of one sample did not detect any other editing events, or indels at this site. Productive edits are indicated within boxed area.

B. ABE on Target 2—c.3113C>T. None of the tested sgRNA/ABE combinations could place the target mutation within the optimal base editing window, and thus minimal editing was observed at that site. PS1 produced bystander editing of an adenine at the 5th nucleotide of the target protospacer, at higher rates than at the target mutation, resulting in a missense amino acid change.

C. CBE on Target 3—c.1622T>C. Moderate editing (up to 15%) was observed at this site with sgRNA-mut3-PS1+Sp (NRTH) BE4max, which places the target nucleotide at position 6 of the protospacer. Bystander editing at position 3 results in a missense amino acid change. If this missense change is deleterious to ABCA4 expression of function, then precise correction of the target mutation will be required.

D. ABE on Target 4—c.5714+5G>A. Highest editing (up to 38%) was observed at this site with sgRNA-mut4-PS2+Sp (NGG) ABE8e, which places the target nucleotide at position 6 of the protospacer. Bystander editing at position 3 and 10 result in changes to the intronic sequence. Deep sequencing of one sample did not detect any other edits or indels at this target site. Presumed productive edits are indicated within boxed area. Intronic changes should be assayed for any impact on gene expression.

E. ABE on Target 5—c.4234C>T. Highest editing (around 40%) was observed at this site with sgRNA-mut5-PS1 or 2+Sp (NGG) ABE8e, which places the target nucleotide at position 7 or 8, respectively, of the protospacer. High rates of bystander editing at positions 2 (3) and 13 (14) result in missense codon changes. sgRNA-mut5-PS1 or 2+Sp (NGG) ABE7.10 exhibited lower editing efficiency at the target nucleotide (around 24%), but very low editing at bystander sites, resulting in higher productive editing overall. Deep sequencing of one sample detected similar on-target editing (24%), and also very low bystander edits (<1%). Productive edits are indicated within boxed region.

F. ABE on Target 6—c.1957C>T. Highest editing (around 35%) was observed at this site with sgRNA-mut6-PS1+Sp (NG) ABE8e, which places the target nucleotide at position 6 of the protospacer. High rates of bystander editing at positions 3 and 11 result in missense codon changes. sgRNA-mut6-PS1+Sp (NGA) ABE8e exhibited lower editing efficiency at the target nucleotide (around 24%), but very low editing at bystander sites, resulting in higher productive editing overall. Deep sequencing of one sample detected similar on-target editing (around 23%), and also very low bystander edits (<1%). Productive edits are indicated within boxed region.

G. ABE on Target 7—c.3322C>T. Highest editing (around 35%) was observed at this site with sgRNA-mut7-PS1 Sp (NG) ABE8e, which places the target nucleotide at position 7 of the protospacer. High rates of bystander editing at positions 4 and 10 result in missense codon changes. sgRNA-mut7-PS1+Sp (NGA) ABE8e exhibited lower editing efficiency at the target nucleotide (around 20%), but very low editing at bystander sites, resulting in higher productive editing overall. Deep sequencing of one sample detected similar on-target editing, and also bystander edits (around 8%). Productive edits are indicated within boxed region (17.3%).

H. ABE on Target 8—c.4771G>A. Highest editing (around 35%) was observed at this site with sgRNA-mut8-PS1 Sp (NGG) ABE8e, which places the target nucleotide at position 4 of the protospacer. Bystander editing at positions 9 and 13 result in nucleotide changes in the intronic sequence. Deep sequencing of one sample detected similar on-target editing (28.1%), and no other nucleotide changes or indels. Productive edits are indicated within boxed region.

I. ABE on Target 9—c.5693G>A. No significant editing was observed on Target 9 with sgRNA-mut8-PS1+ABE8e.

### DETAILED DESCRIPTION

New evolutions of CRISPR/Cas9 based genome editing technology are broadening the scope of potential therapies for previously untreatable diseases. Base editing is able to induce permanent C to T, or A to G, base conversions in the genome at high efficiencies, without double strand breaks (DSBs) [10-14].

The CRISPR based genome editing system has been shown to be an efficient gene-targeting technology in mammalian cells and many organisms [15-18], and can alter or correct genomic sequences by introducing double stranded breaks (DSBs) near the targeting site, which are then repaired through the non-homologous end joining (NHEJ) pathway or, if a DNA template is also supplied, via the Homology Directed Repair (HDR) pathway [19, 20]. While genome editing approaches have great potential, existing genome editors that create double strand breaks (DSBs) in the target DNA have potential limitations, including the risk of genome toxicity [21] from off-target insertions and deletions (indels) created during cellular repair of the DSBs [22, 23]. Furthermore, since the NHEJ pathway is favored over HDR for DNA repair, destructive indels are more often formed than productive edits, and thus rates of correction by HDR are currently too low to be therapeutically relevant [24].

In contrast, base editing technology utilizes a Cas9 nickase (nCas9) or a dead Cas9 (dCas9) nuclease linked with a cytidine or an adenine deaminase (designated as CBE and ABE respectively) to induce a permanent C to T, or A to G, base conversion in the genome with very rare indel formation [10-14]. The guide RNA/BE complex binds DNA at the target site, and catalyzes the base change within a narrow window of the protospacer sequence (optimally position 4-8 for CBE, and slightly broader for ABE). PAMs on either strand that place the mutation within this window, enable reversion of all 4 transition mutations C>T, G>A, A>G and T>C.

Base editing efficiencies of 30-60% have been reported in in vitro studies [13], and are functional in vivo [10, 12, 14, 25-27]. In a previous study, in vivo editing rates of up to 48% in photoreceptor cells were observed after sub-retinal delivery of a split BE:sgRNA complex with AAV-PHP.B or AAV-Anc80 at the proof-of-concept locus DNMT1 [28]. These editing efficiencies are comparable to those reported in preclinical data for EDIT-101, an AAV treatment for Leber congenital amaurosis that relies on Cas9 nuclease activity [29].

ABCA4 is a highly polymorphic gene with over 900 disease-associated variants identified to date. As described herein, known pathogenic variants of ABCA4 that cause STGD1 were analyzed, and a panel of the most common mutations that could be potential targets for base editing strategies was identified. A series of Base Editor (BE) components was designed and systematically tested in cells, to identify optimal BE/sgRNA candidates for correcting the most common ABCA4 mutations.

Possible BEs were identified for the 11 most common ABCA4 mutations and tested in vitro in transient transfections. Up to 50% editing was seen on target mutations that were placed in the optimal editing window. Target 1 (c.5882G>A, p.G1961E), which is the most common ABCA4 mutation, proved to be an excellent candidate for correction by base editing, with up to 50% correction of the target mutation observed. Editing at the sole bystander nucleotide resulted in a synonymous change, and indels or other editing events were not observed at this threshold of sensitivity.

Up to 38% correction was observed for Target 4 (c.5714+5G>A), and similarly for Target 8 (c.4771G>A). Bystander editing was also observed at each target, at 2 intronic nucleotides within each protospacer. A preliminary assessment of predicted splicing effects was conducted in Alamut, and found they are unlikely to affect the normal transcript. This prediction could be tested in splicing assays.

For Targets 5 (c.4234C>T), 6 (c.1957C>T), and 7 (c.3322C>T), multiple sgRNA/BE combinations were tested, and a combination for each that maximized correction at the target nucleotide, while minimizing bystander edits that resulted in missense mutations, was identified. Preliminary in silico assessment of these missense changes did not find any reports of pathogenicity, but each change should be tested further.

Lower or no editing was observed for the other 5 targets. This may be improved by optimization of the transfection conditions, or with the advent of higher efficiency base editors.

To date, there are many studies reporting that BE can achieve some of the highest rates of correction over other CRISPR/Cas9 based editing strategies such as Prime Editing, with favorable off-target effects and indel rates. We have identified several sgRNA/BE combinations that could be developed into effective, single administration therapeutic interventions.

### BE Compositions

To allow the direct installation or correction of point mutations in living cells, engineered proteins that directly convert a target base pair to a different base pair without creating double-stranded DNA breaks, known as base editing, have recently been developed.

### BE

The base editors (e.g., cytidine base editors and adenine base editors) used in the present disclosure are fusion proteins containing a deaminase (e.g., cytosine or adenosine deaminase), and a napDNAbp. Base Editors are known in the art and include cytosine base editors (CBE) and adenine base editors (ABE) that allow for the targeted deamination of cytosines and adenines, respectively, that are exposed on ssDNA by RNA-guided CRISPR-Cas proteins [10, 11, 30-33]. Cytosine base editors (CBEs), such as BE3 or BE4max, catalyze the conversion of target C⋅G base pairs to T⋅A, while adenine base editors (ABEs), such as ABE7.10, ABEmax, or ABE8, convert target A⋅T base pairs to G⋅C. Base editing with canonical base editors requires the presence of a PAM located approximately 15±2 base pairs from the target nucleotide(s). Each programmable DNA-binding protein (napDNAbp) domain recognizes a different PAM sequence. Reference is made to Komor, A. C. et al., Improved base excision repair inhibition and bacteriophage Mu Gam protein yields C:G-to-T:A base editors with higher efficiency and product purity, Sci Adv 3 (2017); Rees, H. A. et al., Improving the DNA specificity and applicability of base editing through protein engineering and protein delivery, Nat. Commun. 8, 15790 (2017); U.S. Patent Application Publication No. 2018/0073012, published Mar. 15, 2018; U.S. Patent Application Publication No. 2017/0121693, published May 4, 2017; International Publication No. WO 2017/070633, published Apr. 27, 2017; and U.S. Patent Publication No. 2015/0166980, published Jun. 18, 2015, U.S. Pat. No. 9,840,699, issued Dec. 12, 2017; and U.S. Pat. No. 10,077,453, issued Sep. 18, 2018, each of which is incorporated herein by reference. Split ABEs or CBEs can also be used, see, e.g., [28, 34-36].

In exemplary embodiments, the presently disclosed compositions, kits, and methods make use of base editors with diverse Cas homologs (e.g., BE3 or BE4max-SpCas9WT, SpCas9VRQR, SaCas9WT, SaCas9KKH, Cpf1; ABE7.10, or ABEmax, or ABE8eSpCas9WT, SpCas9VRQR, SpCas9NG, SpCas9NRTH, SpCas9NRRH, SpCas9MQKSER, SaCas9WT, SaCas9KKH) to install targeted edits in the ABCA4 gene. See, e.g., Kleinstiver et al., Nat Biotechnol. 2015 December; 33(12):1293-1298; Walton et al., Science. 2020 Apr. 17; 368(6488):290-29; Kleinstiver et al., Nature. 2015 Jul. 23; 523(7561):481-5; Bharathkumar et al., Mol Biotechnol. 2022 April; 64(4):355-372; Molla and Yang, Trends Biotechnol. 2019 October; 37(10):1121-1142.

Described herein are compositions comprising sgRNAs comprising a Protospacer Sequence as described in Table 2, e.g., comprising one of SEQ ID NOs:6 to 37, and optionally a corresponding base editor (ABE or CBE, e.g., as indicated in Table 2 or 3), referred to herein as ABCA4 genome editing systems. The term “ABCA4 genome editing system” refers to a system having RNA-guided DNA base editing activity. Genome editing systems of the present disclosure include a gRNA and base editor. These two components form a complex that is capable of associating with a specific nucleic acid sequence in a cell and editing the DNA in or around that nucleic acid sequence. The ABCA4 genome editing system compositions can include ribonucleoprotein complexes comprising the sgRNA/BE; or can include nucleic acids encoding the sgRNA/BE; or can include the sgRNA and nucleic acids encoding the BE. Such nucleic acids can include mRNA or cDNA encoding the proteins, and the nucleic acids can be naked or in an expression vector, e.g., comprising a regulatory sequence such as a promoter that drives expression of the protein. In some embodiments, the nucleic acids encoding the BE, and sgRNA, are expression vectors, e.g., viral vectors.

In some embodiments the BE sequence is modified, e.g., to include one or two nuclear localization sequences (NLSs) (e.g., PKKKRKV (SEQ ID NO:1) at the C- and N-termini of the fusion protein, and a mini-polyadenylation signal (or Poly-A sequence). An exemplary NLS is SV40 large T antigen NLS (PKKKRRV (SEQ ID NO:2)) and nucleoplasmin NLS (KRPAATKKAGQAKKKK (SEQ ID NO:3)). Other NLSs are known in the art; see, e.g., [37, 38]. An exemplary polyadenylation signal is TAGCAATAAAGGATCGTTTATTTTCATTGGAAGCGTGTG TTGGTTTTTTGATCAGGCGCG (SEQ ID NO:4)).

### Expression Constructs and Viral Delivery Systems

Expression constructs comprising sequences encoding ABCA4 genome editing system components as described herein (BE, and sgRNA, optionally wherein the BE is split) can include viral vectors, including recombinant retroviruses, adenovirus, adeno-associated virus, lentivirus, and herpes simplex virus-1, or recombinant bacterial or eukaryotic plasmids. Suitable expression constructs can include: a coding region; a promoter sequence, e.g., a promoter sequence that restricts expression to a selected cell type, a conditional promoter, or a strong general promoter; an enhancer sequence; untranslated regulatory sequences, e.g., a 5′untranslated region (UTR), a 3′UTR; a polyadenylation site; and/or an insulator sequence. Such sequences are known in the art, and the skilled artisan would be able to select suitable sequences. See, e.g., Current Protocols in Molecular Biology, Ausubel, F. M. et al. (eds.) Greene Publishing Associates, (1989), Sections 9.10-9.14; Vancura (ed.), Transcriptional Regulation: Methods and Protocols (Methods in Molecular Biology (Book 809)) Humana Press; 2012 edition (2011) and other standard laboratory manuals. In some embodiments, the expression construct is capable of directing expression of the nucleic acid preferentially in a particular cell type (e.g., tissue-specific regulatory elements are used to express the nucleic acid in retinal photoreceptor cells).

Also described herein are ABCA4 genome editing systems, including a sgRNA/BE, that can be administered to a subject in need thereof. The systems can include, e.g., a viral delivery vector, e.g., preferably an adeno-associated virus (AAV) vector that comprises sequences encoding the sgRNA/BE (as noted above, the BE can be split, and encoded across two AAV). Adeno-associated virus is a naturally occurring defective virus that requires another virus, such as an adenovirus or a herpes virus, as a helper virus for efficient replication and a productive life cycle. (For a review see [39]. AAV vectors efficiently transduce various cell types and can produce long-term expression of transgenes in vivo. AAV vectors have been extensively used for gene augmentation or replacement and have shown therapeutic efficacy in a range of animal models as well as in the clinic; see, e.g., [40-42]. AAV vectors containing as little as 300 base pairs of AAV can be packaged and can produce recombinant protein expression. For example, AAV2, AAV5, AAV2/5, AAV2/8 and AAV2/7 vectors have been used to introduce DNA into photoreceptor cells (see, e.g., [43-45]. In some embodiments, the AAV vector can include (or include a sequence encoding) an AAV capsid polypeptide described in PCT/US2014/060163; for example, a virus particle comprising an AAV capsid polypeptide having an amino acid sequence selected from the group consisting of SEQ ID NOs: 1, 3, 5, 7, 9, 11, 13, 15, and 17 of PCT/US2014/060163, and an sgRNA/BE as described herein. In some embodiments, the AAV capsid polypeptide is an Anc80 polypeptide, e.g., Anc80L27; Anc80L59; Anc80L60; Anc80L62; Anc80L65, Anc80L33; Anc80L36; or Anc80L44. In some embodiments, the AAV incorporates inverted terminal repeats (ITRs), e.g., derived from the AAV2 serotype. Exemplary left and right ITRs are presented in Table 6 of WO 2018/026976. It should be noted, however, that numerous modified versions of the AAV2 ITRs are used in the field, and the ITR sequences shown below are exemplary and are not intended to be limiting. Modifications of these sequences are known in the art, or will be evident to skilled artisans, and are thus included in the scope of this disclosure.

BE expression can be driven by a promoter known in the art. In some embodiments, expression is driven by one of three promoters: cytomegalovirus (CMV), elongation factor-1 (EFS), or human g-protein receptor coupled kinase-1 (hGRK1), which is specifically expressed in retinal photoreceptor cells. Nucleotide sequences for each of these promoters are provided in Table 5 of WO 2018/026976. Modifications of these sequences may be possible or desirable in certain applications, and such modifications are within the scope of this disclosure.

Expression of the sgRNA in the AAV vector can be driven by a promoter known in the art. In some embodiments, a polymerase III promoter, such as a human U6 promoter. An exemplary U6 promoter sequence is presented below:

(SEQ ID NO: 5)
AAGGTCGGGCAGGAAGAGGGCCTATTTCCCATGATTCCTTCATATTTGC
ATATACGATACAAGGCTGTTAGAGAGATAATTAGAATTAATTTGACTGT
AAACACAAAGATATTAGTACAAAATACGTGACGTAGAAAGTAATAATTT
CTTGGGTAGTTTGCAGTTTTAAAATTATGTTTTAAAATGGACTATCATA
TGCTTACCGTAACTTGAAAGTATTTCGATTTCTTGGCTTTATATATCTT
GTGGAAAGGACGAAACACC.

In some embodiments, the nucleic acid or AAV vector shares at least 80%, 85%, 90%, 95%, 96%, 97%, 98%, 99% or greater sequence identity with one of the nucleic acids or AAV vectors recited above.

The AAV genomes described above can be packaged into AAV capsids (for example, AAV5 capsids), which capsids can be included in compositions (such as pharmaceutical compositions) and/or administered to subjects. An exemplary pharmaceutical composition comprising an AAV capsid according to this disclosure can include a pharmaceutically acceptable carrier such as balanced saline solution (BSS) and one or more surfactants (e.g., Tween 20) and/or a thermosensitive or reverse-thermosensitive polymer (e.g., pluronic). Other pharmaceutical formulation elements known in the art may also be suitable for use in the compositions described here.

### Methods of Treatment

The methods described herein include methods for the treatment of disorders associated with mutations in ABCA4 in a subject. Methods for identifying subjects who can be treated by the present methods are known in the art.

In some embodiments, the disorder is Stargardt's disease (STGD1). Subjects with Stargardt's disease typically have bilateral central visual loss, including dyschromatopsia and central scotomata, with macular atrophy and yellow-white flecks at the posterior pole of the retinal pigment epithelium (RPE), with presentation typically in childhood, early adulthood, or later adulthood. The disease is associated with slow, progressive loss of retinal structure and function. A diagnosis can be made by a skilled healthcare provided, e.g., using retinal imaging (e.g., with fundus autofluorescence (FAF) or spectral-domain optical coherence tomography (SD-OCT)) or electrophysiological assessment (e.g., using pattern, full-field and multifocal electroretinography)), and confirmed by detection of a mutation in an ABCA4 allele. See, [46]. Generally, the methods include administering a therapeutically effective amount of an ABCA4 genome editing system as described herein, to a subject who is in need of, or who has been determined to be in need of, such treatment.

As used in this context, to “treat” means to ameliorate at least one symptom of the disorder associated with mutations in the ABCA4 gene. Often, these mutations result in progressive loss of sight; thus, a treatment comprising administration of a therapeutic gene editing system as described herein can result in a reduction in visual impairment; a reduction in the rate of progression of vision loss; and/or a return or approach to normal vision. Vision can be tested using known methods, e.g., electroretinogram, optical coherence tomography, videonystagmography, and audiology testing.

The methods can be used to treat any subject (e.g., a mammalian subject, preferably a human subject) who has a mutation in one or both alleles of ABCA4. A number of pathological mutations have been identified (see Refs. [47-50]). As used herein, an “allele” is one of a pair or series of genetic variants of a polymorphism (also referred to as a mutation) at a specific genomic location. As used herein, “genotype” refers to the diploid combination of alleles for a given genetic polymorphism. A homozygous subject carries two copies of the same allele and a heterozygous subject carries two different alleles. Methods for identifying subjects with such mutations are known in the art; see, e.g., [51-53]. For example, gel electrophoresis, capillary electrophoresis, size exclusion chromatography, sequencing, and/or arrays can be used to detect the presence or absence of the allele or genotype. Amplification of nucleic acids, where desirable, can be accomplished using methods known in the art, e.g., PCR. In one example, a sample (e.g., a sample comprising genomic DNA), is obtained from a subject. The DNA in the sample is then examined to identify or detect the presence of an allele or genotype as described herein. The allele or genotype can be identified or determined by any method described herein, e.g., by Sanger sequencing or Next Generation Sequencing (NGS). Other methods can include hybridization of the gene in the genomic DNA, RNA, or cDNA to a nucleic acid probe, e.g., a DNA probe (which includes cDNA and oligonucleotide probes) or an RNA probe. The nucleic acid probe can be designed to specifically or preferentially hybridize with a particular mutation (also referred to as a polymorphic variant).

Other methods of nucleic acid analysis can include direct manual sequencing (Church and Gilbert, Proc. Natl. Acad. Sci. USA 81:1991-1995 (1988); Sanger et al., Proc. Natl. Acad. Sci. USA 74:5463-5467 (1977); Beavis et al., U.S. Pat. No. 5,288,644); automated fluorescent sequencing; single-stranded conformation polymorphism assays (SSCP) (Schafer et al., Nat. Biotechnol. 15:33-39 (1995)); clamped denaturing gel electrophoresis (CDGE); two-dimensional gel electrophoresis (2DGE or TDGE); conformational sensitive gel electrophoresis (CSGE); denaturing gradient gel electrophoresis (DGGE) (Sheffield et al., Proc. Natl. Acad. Sci. USA 86:232-236 (1989)); denaturing high performance liquid chromatography (DHPLC, Underhill et al., Genome Res. 7:996-1005 (1997)); infrared matrix-assisted laser desorption/ionization (IR-MALDI) mass spectrometry (WO 99/57318); mobility shift analysis (Orita et al., Proc. Natl. Acad. Sci. USA 86:2766-2770 (1989)); restriction enzyme analysis (Flavell et al., Cell 15:25 (1978); Geever et al., Proc. Natl. Acad. Sci. USA 78:5081 (1981)); quantitative real-time PCR (Raca et al., Genet Test 8(4):387-94 (2004)); heteroduplex analysis; chemical mismatch cleavage (CMC) (Cotton et al., Proc. Natl. Acad. Sci. USA 85:4397-4401 (1985)); RNase protection assays (Myers et al., Science 230:1242 (1985)); use of polypeptides that recognize nucleotide mismatches, e.g., E. coli mutS protein; allele-specific PCR, and combinations of such methods. See, e.g., Gerber et al., U.S. Patent Publication No. 2004/0014095 which is incorporated herein by reference in its entirety.

Compositions comprising ABCA4 genome editing system components, e.g., proteins or nucleic acids, e.g., AAV vectors, as described herein can be administered to subjects by any suitable means, including without limitation injection, for example, subretinal injection. The concentration of the active compounds vector within the composition is selected to ensure, among other things, that a sufficient dose is administered to the retina of the subject, taking account of dead volume within the injection apparatus and the relatively limited volume that can be safely administered. Suitable doses for AAV may include, for example, 1×10¹¹ viral genomes (vg)/mL, 2×10¹¹ viral genomes (vg)/mL, 3×10¹¹ viral genomes (vg)/mL, 4×10¹¹ viral genomes (vg)/mL, 5×10¹¹ viral genomes (vg)/mL, 6×10¹¹ viral genomes (vg)/mL, 7×10¹¹ viral genomes (vg)/mL, 8×10¹¹ viral genomes (vg)/mL, 9×10¹¹ viral genomes (vg)/mL, 1×10¹² vg/mL, 2×10¹² viral genomes (vg)/mL, 3×10¹² viral genomes (vg)/mL, 4×10¹² viral genomes (vg)/mL, 5×10¹² viral genomes (vg)/mL, 6×10¹² viral genomes (vg)/mL, 7×10¹² viral genomes (vg)/mL, 8×10¹² viral genomes (vg)/mL, 9×10¹² viral genomes (vg)/mL, 1×10¹³ vg/mL, 2×10¹³ viral genomes (vg)/mL, 3×10¹³ viral genomes (vg)/mL, 4×10¹³ viral genomes (vg)/mL, 5×10¹³ viral genomes (vg)/mL, 6×10¹³ viral genomes (vg)/mL, 7×10¹³ viral genomes (vg)/mL, 8×10¹³ viral genomes (vg)/mL, or 9×10¹³ viral genomes (vg)/mL. Any suitable volume of the composition may be delivered to the subretinal. In some instances, the volume is selected to form a bleb in the subretinal space, for example 1 microliter, 10 microliters, 50 microliters, 100 microliters, 150 microliters, 200 microliters, 250 microliters, 300 microliters, etc.

Any region of the retina may be targeted, though the fovea (which extends approximately 1 degree out from the center of the eye) may be preferred in certain instances due to its role in central visual acuity and the relatively high concentration of cone photoreceptors there relative to peripheral regions of the retina. Alternatively or additionally, injections may be targeted to parafoveal regions (extending between approximately 2 and 10 degrees off center), which are characterized by the presence of all three types of retinal photoreceptor cells. In addition, injections into the parafoveal region may be made at comparatively acute angles using needle paths that cross the midline of the retina. For instance, injection paths may extend from the nasal aspect of the sclera near the limbus through the vitreal chamber and into the parafoveal retina on the temporal side, from the temporal aspect of the sclera to the parafoveal retina on the nasal side, from a portion of the sclera located superior to the cornea to an inferior parafoveal position, and/or from an inferior portion of the sclera to a superior parafoveal position. The use of relatively small angles of injection relative to the retinal surface may advantageously reduce or limit the potential for spillover of vector from the bleb into the vitreous body and, consequently, reduce the loss of the vector during delivery. In other cases, the macula (inclusive of the fovea) can be targeted, and in other cases, additional retinal regions can be targeted, or can receive spillover doses.

For pre-clinical development purposes, systems, compositions, nucleotides and vectors according to this disclosure can be evaluated ex vivo using a retinal explant system, or in vivo using an animal model such as a mouse, rabbit, pig, nonhuman primate, etc. Retinal explants are optionally maintained on a support matrix, and AAV vectors can be delivered by injection into the space between the photoreceptor layer and the support matrix, to mimic subretinal injection. Tissue for retinal explanation can be obtained from human or animal subjects, for example mouse.

Explants are particularly useful for studying the expression of gRNAs and/or Cas9 following viral transduction, and for studying genome editing over comparatively short intervals. These models also permit higher throughput than may be possible in animal models, and can be predictive of expression and genome editing in animal models and subjects. Small (mouse, rat) and large animal models (such as rabbit, pig, nonhuman primate) can be used for pharmacological and/or toxicological studies and for testing the systems, nucleotides, vectors and compositions of this disclosure under conditions and at volumes that approximate those that will be used in clinic. Because model systems are selected to recapitulate relevant aspects of human anatomy and/or physiology, the data obtained in these systems will generally (though not necessarily) be predictive of the behavior of AAV vectors and compositions according to this disclosure in human and animal subjects.

### EXAMPLES

The invention is further described in the following examples, which do not limit the scope of the invention described in the claims.

### Example 1. Identify and Prioritize a Panel of Known Mutations in ABCA4 that can be Corrected by BE

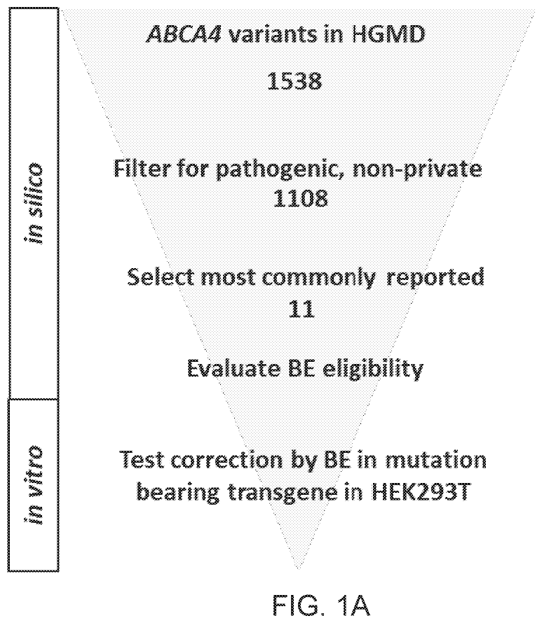

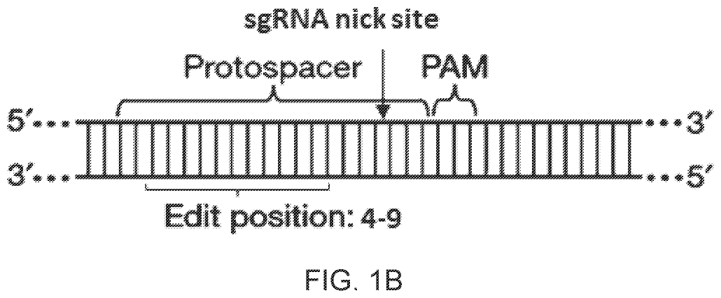

ABCA4 is a highly polymorphic gene with over 900 disease-associated variants identified to date [47, 48, 50, 54]. We analyzed all reported mutations in ABCA4 in The Human Gene Mutation Database (HGMD) (hgmd.cf.ac.uk) and found that around 38% were single nucleotide transition mutations that could theoretically be correct by ABEs or CBEs (FIG. 1A). We filtered the list of the editable mutations to select the eleven most common pathogenic mutations (Table 1). All eleven mutations are single transition mutations, therefore we analyzed the surrounding sequence to identify nearby PAM sites that could be utilized by BEs.

Cas variants with PAMs of NGG, NG, NGA, NRTH, NRCH, NNGRRT and NNNRRT, are currently available for base editing [11, 13, 55]. PAM sites were selected to place the target mutant base within the optimal editing window of 4-9 nt from the 5′ end of the guide sequence (FIG. 1B, Table 2). Other bases within this window, which could be edited in so-called ‘bystander’ effects, were also annotated.

### Example 2. Develop an In Vitro System to Test Editing of Most-Common Mutations

To facilitate the screening of the base editing approaches, we created a synthetic gene comprising tandem aligned DNA fragments, each of which contains the target sites of ABCA4 mutations listed in Table 1. We also included a short sequence from DNMT1, as a positive control site. Each mutant fragment contains 51 bp of ABCA4 sequence flanked by universal priming sequences.

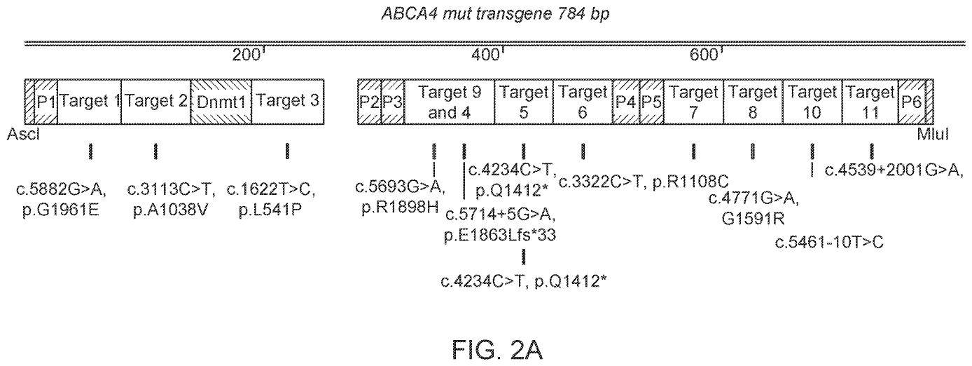

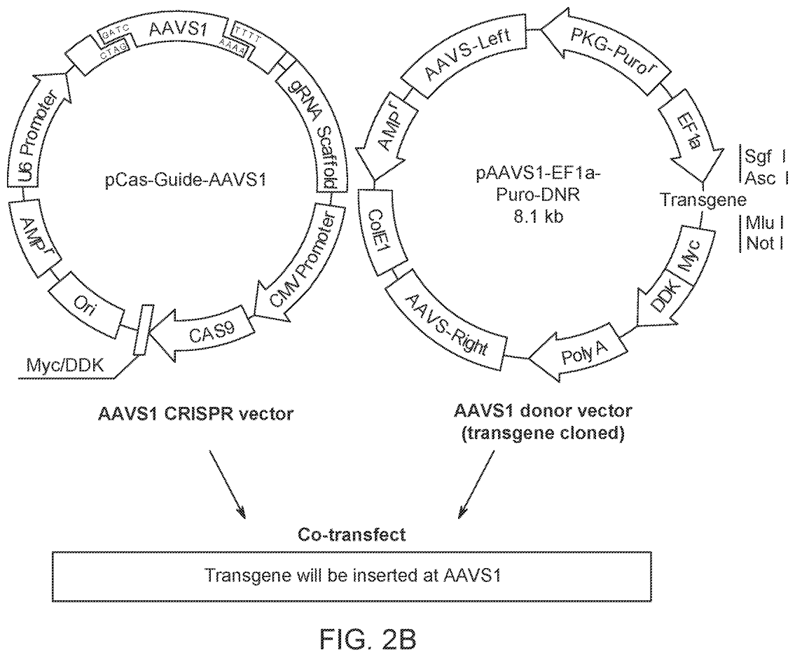

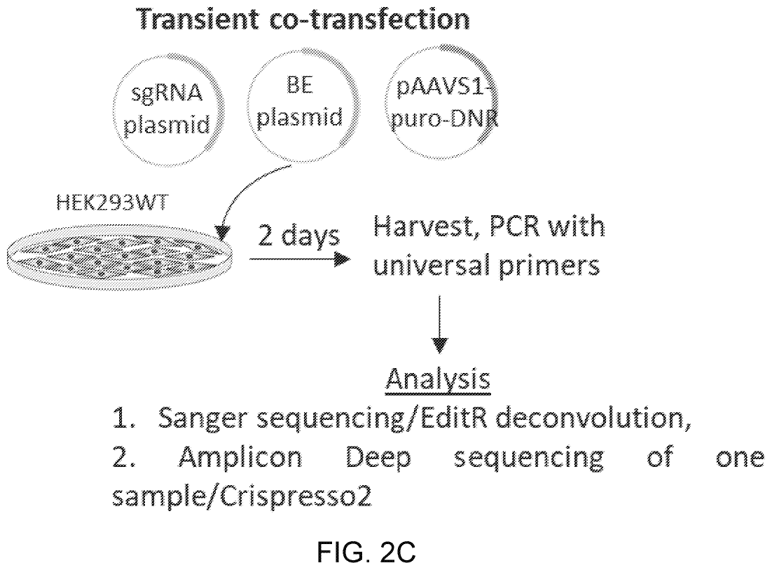

Targets 4 and 9 are separated by 25 nucleotides, and thus were included in a 77 nucleotide fragment. This construct was synthesized as a gblock (FIG. 2A), with restriction sites at each end to facilitate cloning into the MCS of the pAAVS1-puro-DNR plasmid, which contains 550 bp homology arms for the human AAVs1 locus, a puromycin selection cassette under the PGK promoter, and a constitutive EF1a promoter. The experiments were performed as transient transfections, in which the donor plasmid was co-transfected into HEK293T cells along with the relevant BE and sgRNA combination (FIG. 2C.).

A stable cell line can be generated by co-transfecting this vector and a sgRNA/Cas9 plasmid targeting the AAVS1 locus, into HEK293T cells (FIG. 2B). Cells with correct integration of the transgene into the locus by homologous recombination can then be selected according to puromycin resistance, and integration confirmed by locus specific sequencing.

### Example 3. Identify and Optimize Base Editors for Correcting the Common Mutations in ABCA4

Editing constructs for repair of the mutations listed in Table 1 were designed for all available BEs (Table 2), and synthesized according to established techniques. Forward and Reverse oligos encoding the 20 bp protospacer sequence for all targets in (Table 1) were commercially synthesized with appropriate overhangs, and cloned into the expression plasmid U6-sgRNA-CAG-GFP to generate between 1 and 4 sgRNAs for each target. Each sgRNA/BE combination was co-transfected with the ABCA4 mutant transgene donor vector into HEK293T cells with Lipofectamine 3000. Three biological replicates were performed per editing combination. Genomic DNA was extracted from each sample, and the entire transgene was specifically amplified with universal primers, followed by Sanger sequencing across the target mutation, and EditR deconvolution (baseeditr.com/). The percentage of A to G conversions (for ABEs) or C to T conversions (for CBEs) was evaluated for all target nucleotides, as well as for all bystanders within the flanking region, and a qualitative analysis was performed for each to determine if the bystander edit was synonymous, caused a missense change to the coded amino acid, or whether splicing defects were predicted.

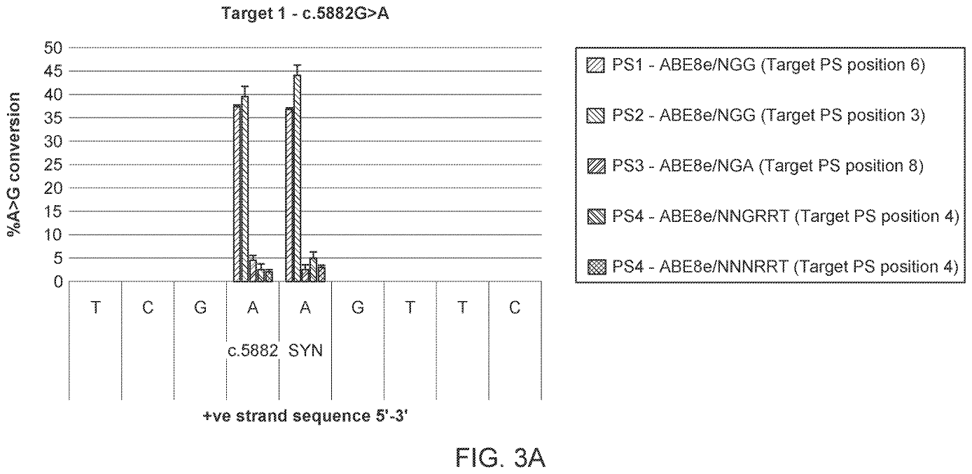

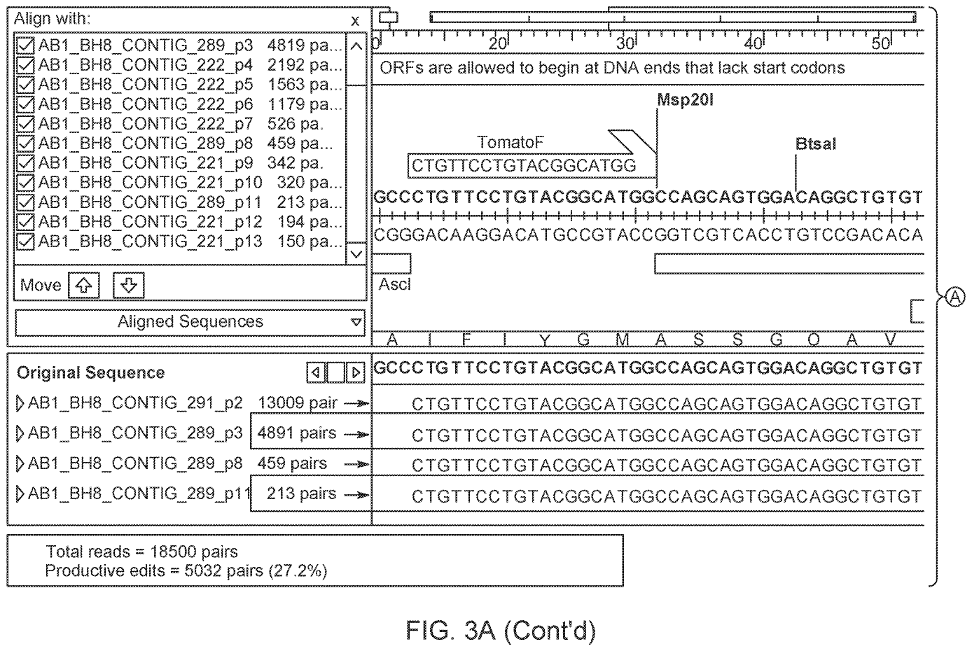

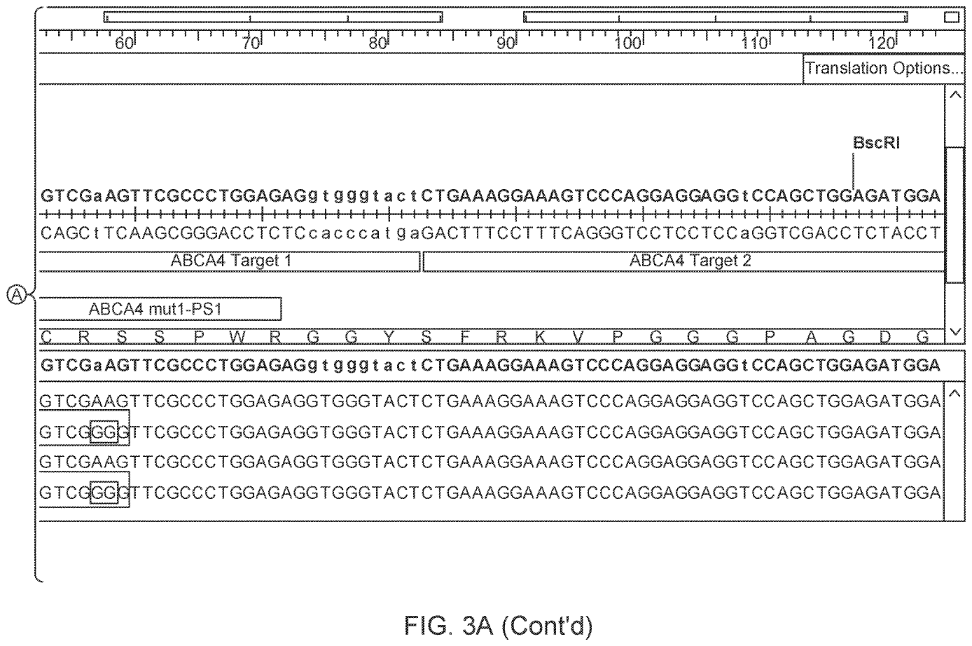

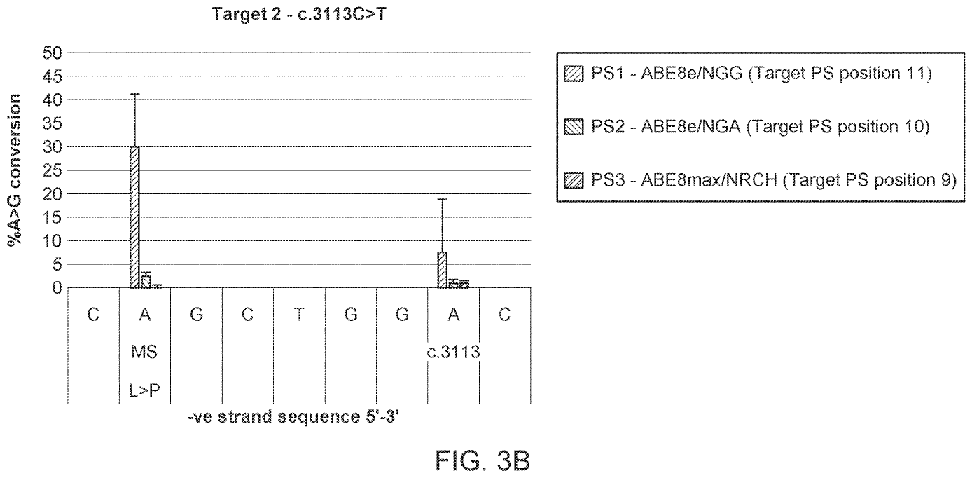

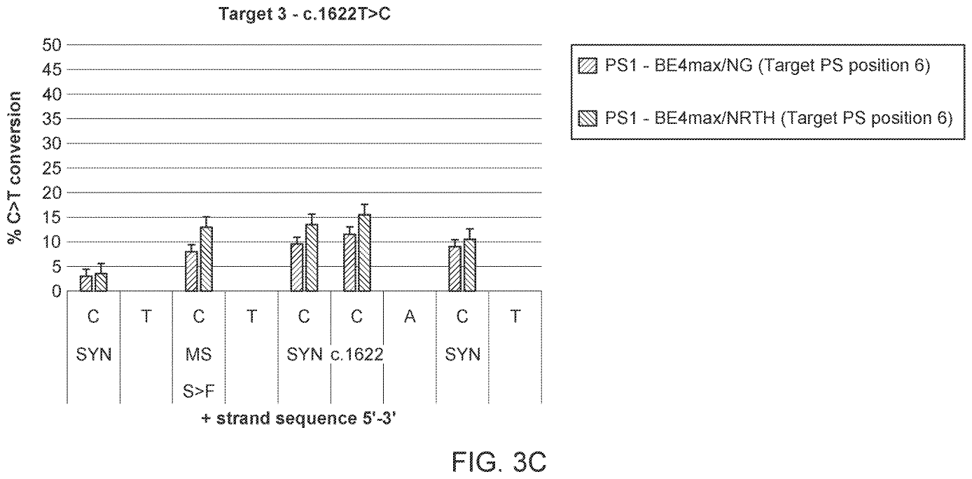

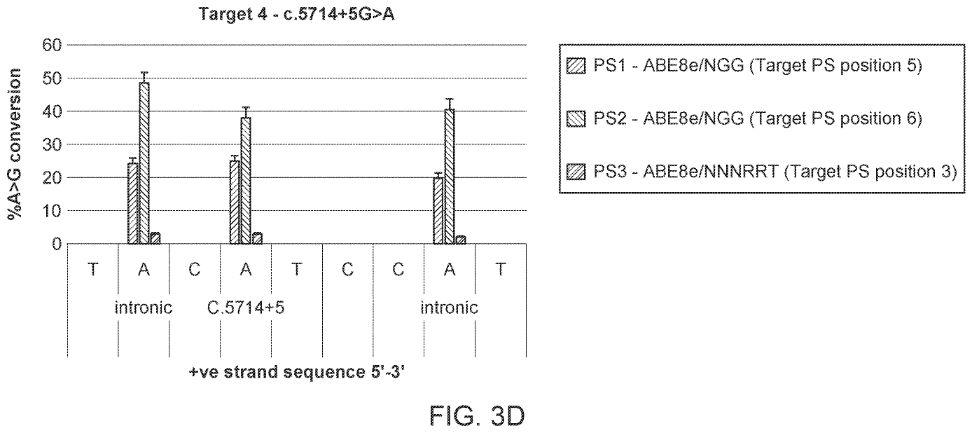

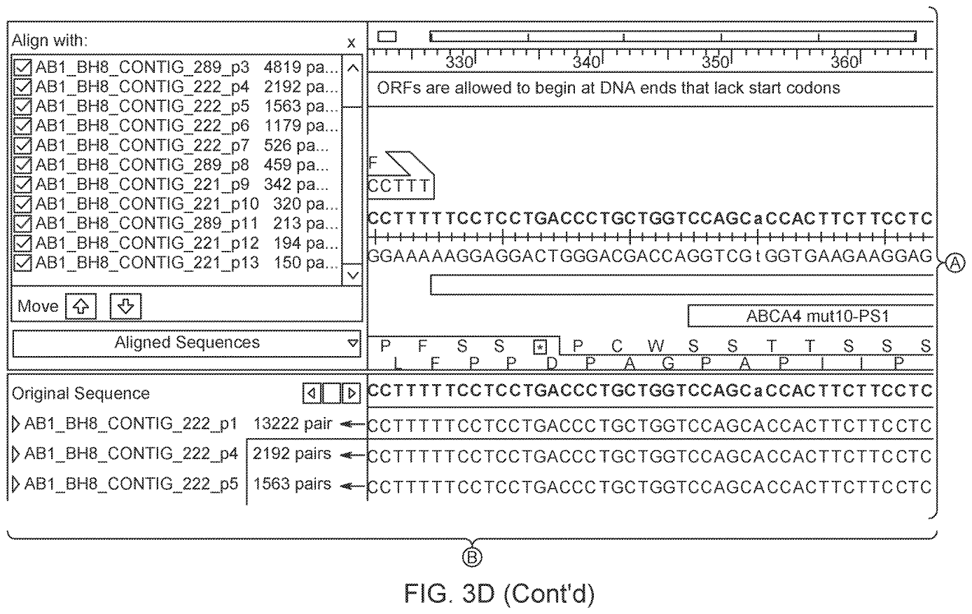

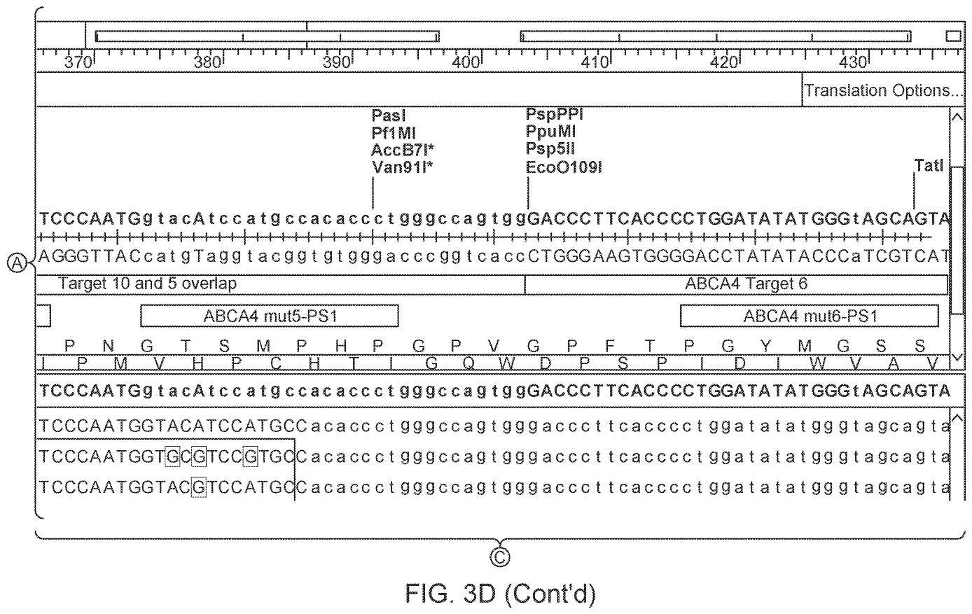

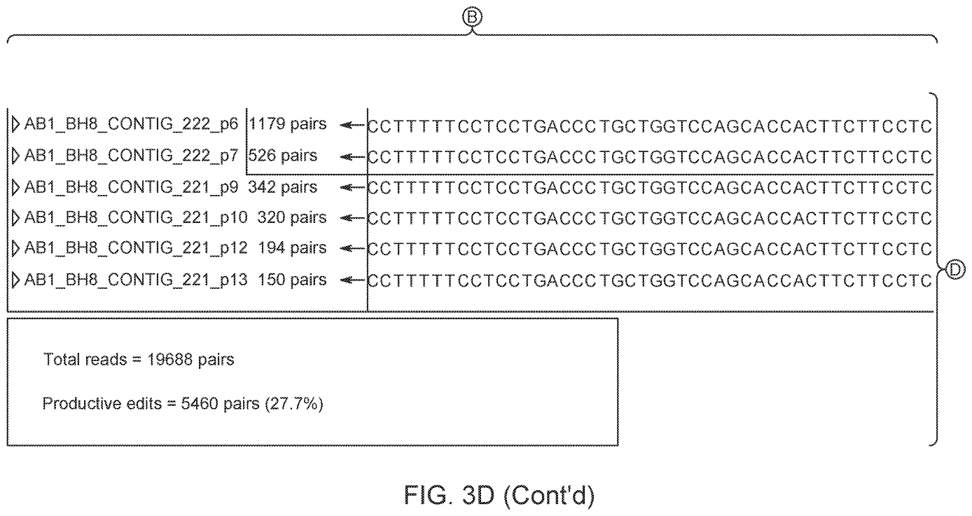

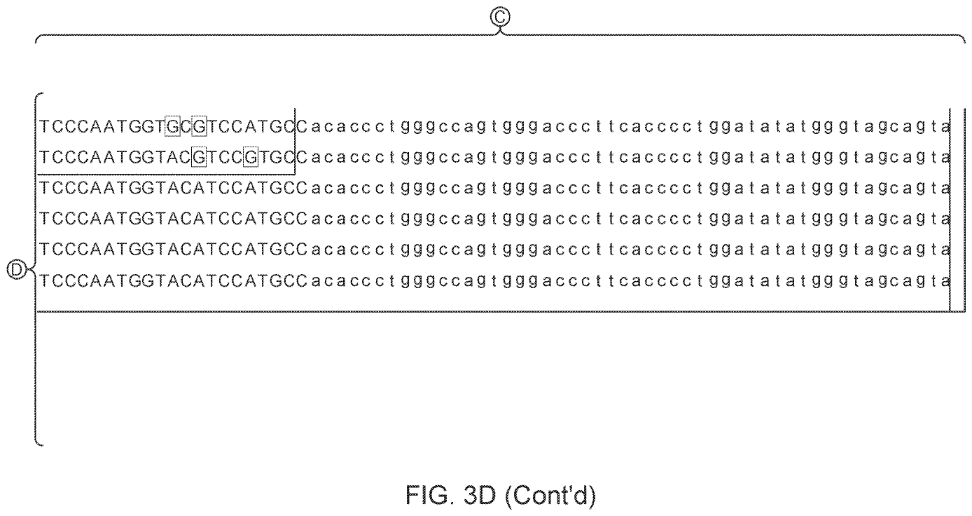

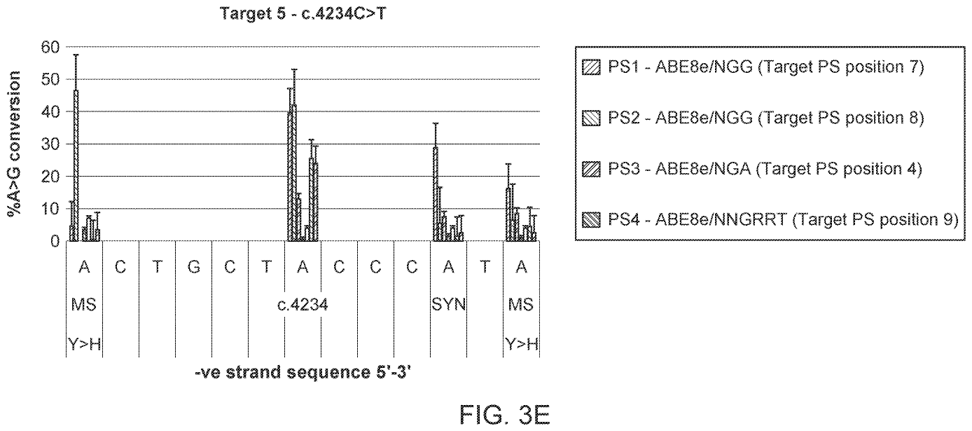

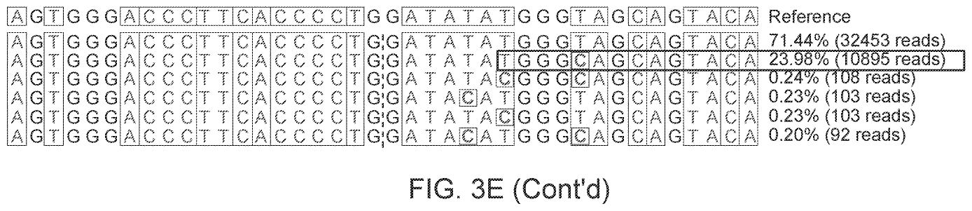

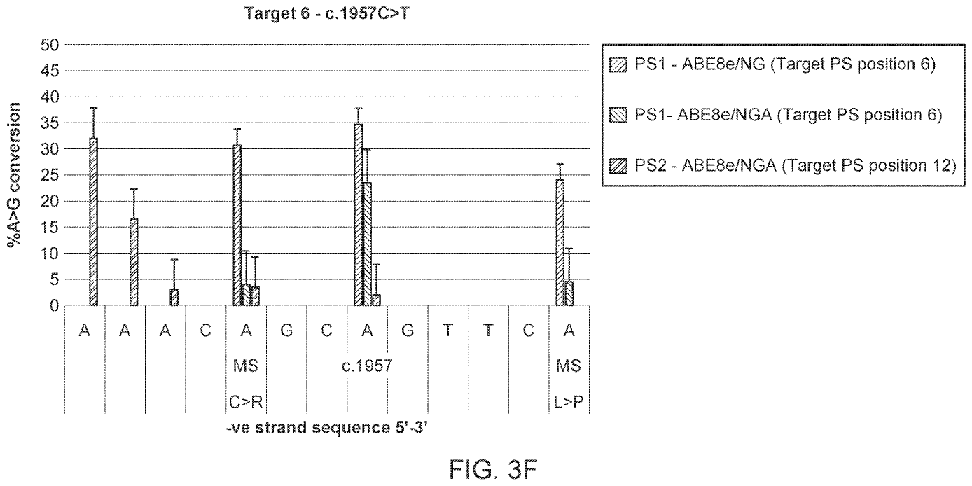

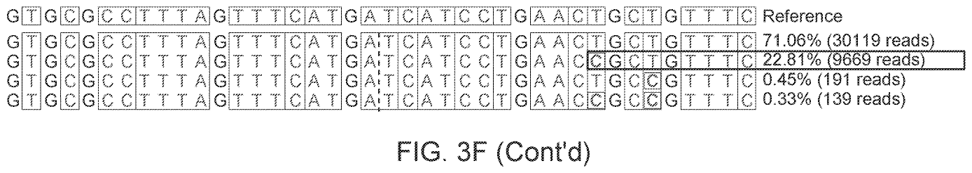

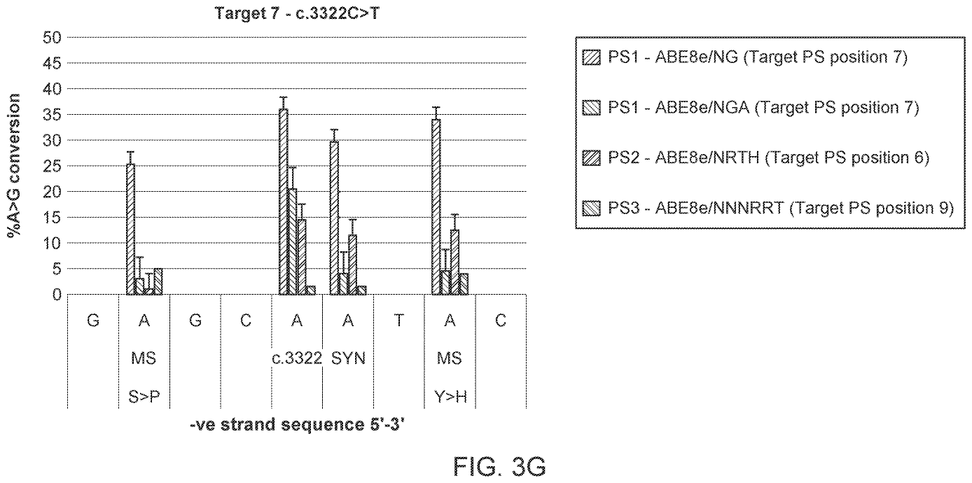

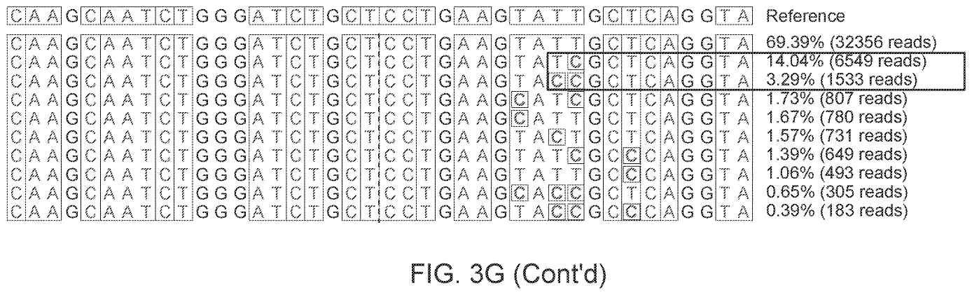

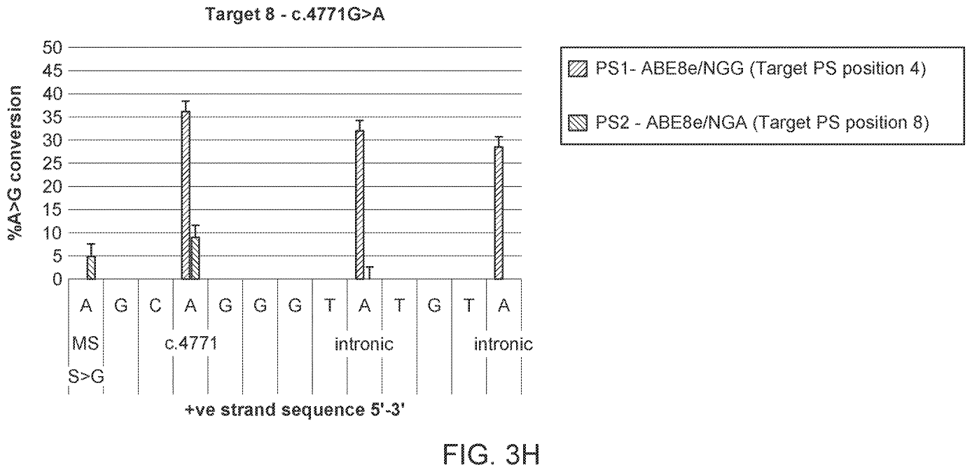

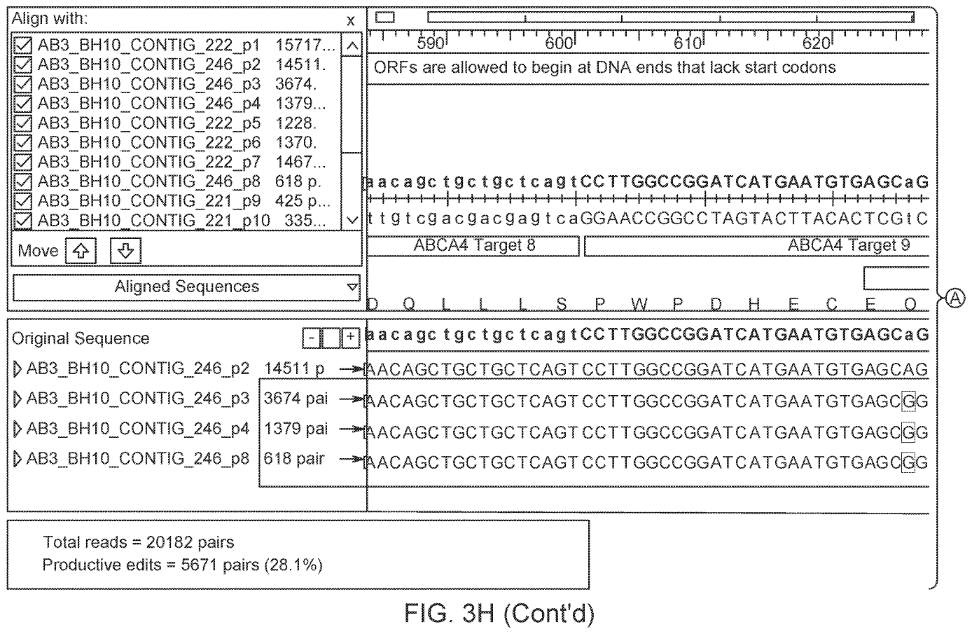

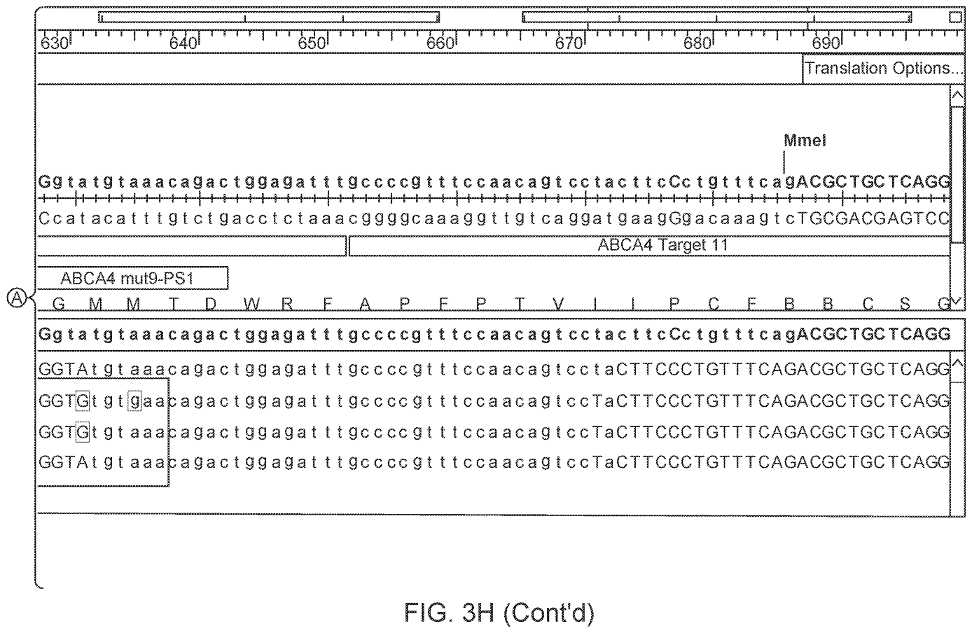

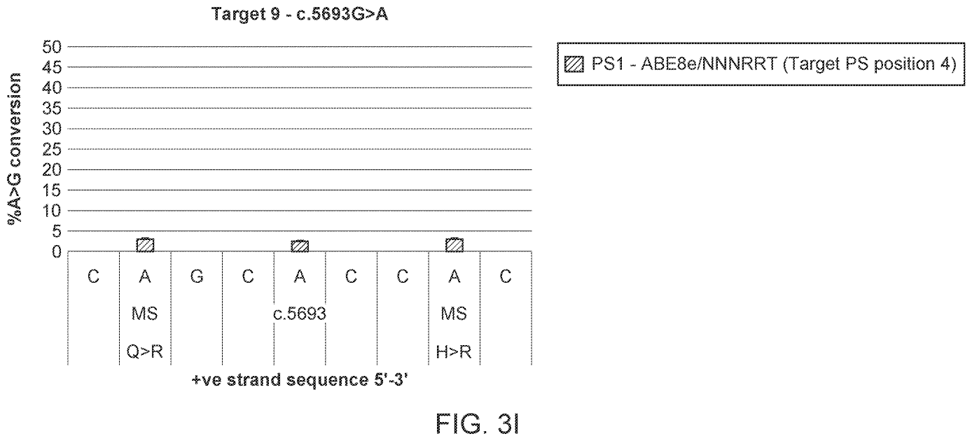

Amplicon next-generation sequencing on a ~250 bp region around each target site was also analyzed using CRISPResso2 [56], for one of the biological replicates, to look for editing events with greater sensitivity than the above described method. Results of all base editing experiments are summarized in Table 3. EditR and Crispresso results for each target are illustrated in FIGS. 3A to I.

TABLE 1
ABCA4 candidate mutations

| | | | | | HGMD | |
|---|---|---|---|---|---|---|
| | | | Position | | relative | HGMD |
| Target | Mutation | Protein | GRCH37 | Exon | frequency | class |
| 1 | c.5882G > A | p.G1961E | 94473807 | 42 | 0.0761 | DM |
| 2 | c.3113C > T | p.A1038V | 94508969 | 21 | 0.0612 | DM |
| 3 | c.1622T > C | p.L541P | 94528806 | 12 | 0.0537 | DM |
| 4 | c.5714 + 5G > A | p.E1863Lfs*33 | 94476351 | 40i | 0.0194 | DM |
| 5 | c.4234C > T | p.Q1412* | 94496571 | 28 | 0.0164 | DM |
| 6 | c.1957C > T | p.R653C | 94526296 | 14 | 0.0075 | DM |
| 7 | c.3322C > T | p.R1108C | 94508323 | 22 | 0.0075 | DM |
| 8 | c.4771G > A | p.G1591R | 94487404 | 33 | 0.0075 | DM? |
| 9 | c.5693G > A | p.R1898H | 94476377 | 40 | 0.006 | DM |
| 10 | c.5461 − 10T > C | p.T1821Dfs*6 | 94476951 | 38i | 0.0343 | DM |
| 11 | c.4539 + 2001G > A | p.R1514Lfs*36 | 94493000 | 30i | ? | DM? |

TABLE 2
Exemplary Base Editing (BE) strategies for ABCA4 candidate mutations
Mutations are indicated in bold, exons in uppercase, introns in lowercase.

| | | | | | Editing | | | | |
|---|---|---|---|---|---|---|---|---|---|
| | | | | | Window | By- | | | SEQ ID |
| Target | Mutation | Base | Strand | PAM | Position | stander | Name | Protospacer Sequence 5′-3′ | NO: |
| 1 | c.5882G > A | ABE | + | NGG | A6 | 7 | ABCA4 | TGTCGAAGTTCGCCCTGGAG | 6. |
| | | | | | | | mut1- | | |
| | | | | | | | PS1 | | |
| | | | | NGG | A3 | 4 | ABCA4 | CGAAGTTCGCCCTGGAGAGg | 7. |
| | | | | | | | mut1- | | |
| | | | | | | | PS2 | | |
| | | | | NGA | A8 | 9 | ABCA4 | TGTGTCGAAGTTCGCCCTGG | 8. |
| | | | | | | | mut1- | | |
| | | | | | | | PS3 | | |
| | | | | NNGRRT | A4 | 5 | ABCA4 | TCGAAGTTCGCCCTGGAGAG | 9. |
| | | | | NNNRRT | | | mut1- | | |
| | | | | | | | PS4 | | |
| 2 | c.311 | ABE | − | NGG | A11 | 5 | ABCA4 | CTCCAGCTGGACCTCCTCCT | 10. |
| | | | | | | | mut2- | | |
| | | | | | | | PS1 | | |
| | | | | NGA | A10 | 4 | ABCA4 | TCCAGCTGGACCTCCTCCTG | 11. |
| | | | | | | | mut2- | | |
| | | | | | | | PS2 | | |
| | | | | NRCH | A9 | 3 | ABCA4 | CCAGCTGGACCTCCTCCTGG | 12. |
| | | | | | | | mut2- | | |
| | | | | | | | PS3 | | |
| 3 | c.162 | CBE | + | NG | C6 | 1, 3, 5, | ABCA4 | CTCTCCACTGGAGGAAAACA | 13. |
| | | | | NRTH | | 8 | mut3- | | |
| | | | | | | | PS1 | | |
| 4 | c.5714 + | ABE | + | NGG | A5 | 3, 9 | ABCA4 | gtacatccatgccacaccct | 14. |
| | 5G > A | | | | | | mut4- | | |
| | | | | | | | PS1 | | |
| | | | | NGG | A6 | 4, 10 | ABCA4 | Ggtacatccatgccacaccc | 15. |
| | | | | | | | mut4- | | |
| | | | | | | | PS2 | | |
| | | | | NNNRRT | A3 | 1, 7, 12 | ABCA4 | acatccatgccacaccctgg | 16. |
| | | | | | | | mut4- | | |
| | | | | | | | PS3 | | |
| 5 | c.4234C > T | ABE | − | NGG | A7 | 1, 11, | ABCA4 | ACTGCTACCCATATATCCAG | 17. |
| | | | | | | 13 | mut5- | | |
| | | | | | | | PS1 | | |
| | | | | NGG | A8 | 2, 12, | ABCA4 | TACTGCTACCCATATATCCA | 18. |
| | | | | | | 14 | mut5- | | |
| | | | | | | | PS2 | | |
| | | | | NGA | A4 | 8, 10, | ABCA4 | GCTACCCATATATCCAGGGG | 19. |
| | | | | | | 12 | mut5- | | |
| | | | | | | | PS3 | | |
| | | | | NNGRRT | A9 | 3, 13, | ABCA4 | GTACTGCTACCCATATATCC | 20. |
| | | | | NNNRRT | | 15 | mut5- | | |
| | | | | | | | PS4 | | |
| 6 | c.1957C > T | ABE | − | NG | A6 | 1, 3, 11 | ABCA4 | ACAGCAGTTCAGGATGATCA | 21 |
| | | | | NGA | | | mut6- | | |
| | | | | | | | PS1 | | |
| | | | | NGA | A12 | 7,9 | ABCA4 | AGGGAAACAGCAGTTCAGGA | 22. |
| | | | | | | | mut6- | | |
| | | | | | | | PS2 | | |
| 7 | c.3322C > T | ABE | − | NG | A7 | 4, 8, 10 | ABCA4 | CTGAGCAATACTTCAGGAGC | 23. |
| | | | | NGA | | | mut7- | | |
| | | | | | | | PS1 | | |
| | | | | NRTH | A6 | 3, 7, 9 | ABCA4 | TGAGCAATACTTCAGGAGCA | 24. |
| | | | | | | | mut7- | | |
| | | | | | | | PS2 | | |
| | | | | NNNRRTA9 | | 1, 6, 10, | ABCA4 | acCTGAGCAATACTTCAGGA | 25. |
| | | | | | | 12 | mut7- | | |
| | | | | | | | PS3 | | |
| 8 | c.477 | ABE | + | NGG | A4 | 1, 9 | ABCA4 | AGCAGGgtatgtaaacagac | 26. |
| | | | | | | | mut8- | | |
| | | | | | | | PS1 | | |
| | | | | NGA | A8 | 5, 13 | ABCA4 | TGTGAGCAGGgtatgtaaac | 27. |
| | | | | | | | mut8- | | |
| | | | | | | | PS2 | | |
| 9 | c.5693G > | ABE | + | NNNRRT | A6 | 3, 9 | ABCA4 | CCAGCACCACTTCTTCCTCT | 28. |
| | | | | | | | mut9- | | |
| | | | | | | | PS1 | | |
| 10 | c.5461− | CBE | + | NRCH | C7 | 3, 6, 8 | ABCA4 | tacttccctgtttcagACGC | 29. |
| | | | | | | | mut10- | | |
| | | | | | | | PS1 | | |
| 11 | c.4539 + | ABE | + | NRCH | A4 | 1 | ABCA4 | AGGACTCCTGTGGCTGCCTC | 30. |
| | 2001 | | | | | | mut11- | | |
| | | | | | | | PS1 | | |
| | | | | NRCH | A10 | 1, 7 | ABCA4 | ATGCTGAGGACTCCTGTGGC | 31. |
| | | | | | | | mut11- | | |
| | | | | | | | PS2 | | |
| 12 | p.P1380L | ABE | − | NGG | A7 | 3,9,12 | | GTAGCCaGGAGCACGATctg | 32. |
| | | | | NGG | A6 | 2, 8, 11 | | TAGCCaGGAGCACGATctgt | 33. |
| | | | | NGA | A5 | 1, 7 | | AGCCaGGAGCACGATctgtg | 34. |
| | | | | | | 10, 13 | | | |
| 13 | p.L2027F | ABE | − | NG | A8 | 7, 10 | | CCTGTGAaCAGCTCATCAAT | 35. |
| | | | | NRTH | A6 | 5, 8 | | TGTGAaCAGCTCATCAATTG | 36. |
| 14 | p.C1490 > Y | ABE | − | NG | A6 | 2, 8, 11 | | CAGGTaCAGCACCAGGGAGA | 37. |
| | | | | NRCH | | | | | |

| | | SEQ ID |
|---|---|---|
| Target | ABCA4 mutation flanked by 50 nucleotides of genomic sequence | NO: |
| 1 | CCAGCAGTGGACAGGCTGTGTGTCGAAGTTCGCCCTGGAGAGgtgggtact | 38. |
| 2 | CTGAAAGGAAAGTCCCAGGAGGAGGTCCAGCTGGAGATGGAAGCCA | 39. |
| | TGTTG | |
| 3 | CAGCTCACCCAACGTGCCCTCTCTCCACTGGAGGAAAACATGTTCTGG | 40. |
| | GCC | |
| 4 | TTCCTCCTGACCCTGCTGGTCCAGCACCACTTCTTCCTCTCCCAATGgta | 41. |
| | catccatgccacaccctgggccagtgg | |
| 5 | GACCCTTCACCCCTGGATATATGGGTAGCAGTACACCTTCTTCAGgtgcg | 42. |
| | c | |
| 6 | ctttagTTTCATGATCATCCTGAACTGCTGTTTCCCTATCTTCATGGTGCT | 43. |
| 7 | AATCTGGGATCTGCTCCTGAAGTATTGCTCAGgtaacagctgctgctcagt | 44. |
| 8 | CCTTGGCCGGATCATGAATGTGAGCAGGgtatgtaaacagactggagattt | 45. |
| 9 | Target 10 and Target 5 overlap | 46. |
| 10 | gccccgtttccaacagtcctacttccctgtttcagACGCTGCTCAGGTTCA | 47. |
| 11 | GTTTGCTTTGCTGTCAATGCTGAGGACTCCTGTGGCTGCCTCCACTCA | 48. |
| | AAA | |
| 12 | tctattctcccacagATCGTGCTCCtGGCTACCTTTGTGTTTTTGGCTCTG | 49 |
| 13 | TCAGTTTGATGCAATTGATGAGCTGUTCACAGGACGAGAACATCTTTA | 50. |
| | CCT | |
| 14 | GTCAACCCTTCACCATCCTGCAGGTaCAGCACCAGGGAGAAGCTCAC | 51. |
| | CATG | |

TABLE 3
Summary of BE results.
Productive editing is calculated as % correction of target
mutation, minus % missense inducing edits at bystander nucleotides.

| | | | | | Editing | | | Target | |
|---|---|---|---|---|---|---|---|---|---|
| | | Base | | | Window | | | Correction | Productive |
| Target | Mutation | Editor | Strand | PAM | Position | BS | Name | % | Editing % |
| 1 | c.5882G > A | ABE8e | + | NGG | A6 | 7 | ABCA4 mut1- | 37 | 37 |
| | | | | | | | PS1 | | |
| | | | | NGG | A3 | 4 | ABCA4 mut1- | 40 | 40 |
| | | | | | | | PS2 | | |
| | | | | NGA | A8 | 9 | ABCA4 mut1- | 5 | 5 |
| | | | | | | | PS3 | | |
| | | | | NNGRRT | A4 | 5 | ABCA4 mut1- | 3 | 3 |
| | | | | NNNRRT | | | PS4 | 2 | 2 |
| 2 | c.3113C > T | ABE8e | − | NGG | A11 | 5 | ABCA4 mut2- | 7.5 | 0 |
| | | | | | | | PS1 | | |
| | | | | NGA | A10 | 4 | ABCA4 mut2- | 1 | 0 |
| | | | | | | | PS2 | | |
| | | ABE8max | | NRCH | A9 | 3 | ABCA4 mut2- | 1 | 0 |
| | | | | | | | PS3 | | |
| 3 | c.1622T > C | BE4max | + | NG | C6 | | ABCA4 mut3- | 11.5 | 3.5 |
| | | | | NRTH | | 1, 3, 5, 8 | PS1 | 15.5 | 2.5 |
| 4 | c.5714 + | ABE8e | + | NGG | A5 | 3, 9 | ABCA4 mut4- | 25 | 25 |
| | 5G > A | | | | | | PS1 | | |
| | | | | NGG | A6 | 4, 10 | ABCA4 mut4- | 38 | 38 |
| | | | | | | | PS2 | | |
| | | | | NNNRRT | A3 | 1, 7, 12 | ABCA4 mut4- | 3 | 3 |
| | | | | | | | PS3 | | |
| 5 | c.4234C > T | ABE8e | − | NGG | A7 | 1, 11, 13 | ABCA4 mut5- | 39.6 | 23.4 |
| | | | | | | | PS1 | | |
| | | | | NGG | A8 | 2, 12, 14 | ABCA4 mut5- | 42 | 0 |
| | | | | | | | PS2 | | |
| | | | | NGA | A4 | 8, 10, 12 | ABCA4 mut5- | 13 | 4.5 |
| | | | | | | | PS3 | | |
| | | | | NNGRRT | A9 | 3, 13, 15 | ABCA4 mut5- | 0.5 | 0 |
| | | | | NNNRRT | | | PS4 | 4 | 0 |
| | | ABE7.10 | − | NGG | A7 | 1, 11, 13 | ABCA4 mut5- | 25.5 | 20.1 |
| | | | | | | | PS1 | | |
| | | | | NGG | A8 | 2, 12, 14 | ABCA4 mut5- | 24 | 20.5 |
| | | | | | | | PS2 | | |
| 6 | c.1957C > T | ABE8e | − | NG | A6 | 1, 3, 11 | ABCA4 mut6- | 34.7 | 4 |
| | | | | NGA | | | PS1 | 23.5 | 19 |
| | | | | NGA | A12 | 7, 9 | ABCA4 mut6- | 2 | 0 |
| | | | | | | | PS2 | | |
| 7 | c.3322C > T | ABE8e | − | NG | A7 | 4, 8, 10 | ABCA4 mut7- | 36 | 2 |
| | | | | NGA | | | PS1 | 20.5 | 16 |
| | | | | NRTH | A6 | 3, 7, 9 | ABCA4 mut7- | 14.5 | 2 |
| | | | | | | | PS2 | | |
| | | | | NNNRRT | A9 | 1, 6, 10, 12 | ABCA4 mut7- | 1.5 | 0 |
| | | | | | | | PS3 | | |
| 8 | c.4771G > A | ABE8e | + | NGG | A4 | 1, 9 | ABCA4 mut8- | 36 | 36 |
| | | | | | | | PS1 | | |
| | | | | NGA | A8 | 5, 13 | ABCA4 mut8- | 9 | 4 |
| | | | | | | | PS2 | | |
| 9 | c.5693G > A | ABE8e | + | NNNRRT | A6 | 3, 9 | ABCA4 mut9- | 2.5 | 0 |
| | | | | | | | PS1 | | |
| 10 | c.5461 − | BE4max | + | NRCH | C7 | 3, 6, 8 | ABCA4 | 0 | 0 |
| | 10T > C | | | | | | mut10-PS1 | | |
| 11 | c.4539 + | ABE8max | + | NRCH | A4 | 1 | ABCA4 | 0 | 0 |
| | 2001G > A | | | | | | mut11-PS1 | | |
| | | | | NRCH | A10 | 1, 7 | ABCA4 | 0 | 0 |
| | | | | | | | mut11-PS2 | | |

### REFERENCES

1. Michaelides, M., D. M. Hunt, and A. T. Moore, The genetics of inherited macular dystrophies. J Med Genet, 2003. 40(9): p. 641-50.
2. Sun, H. and J. Nathans, Stargardt's ABCR is localized to the disc membrane of retinal rod outer segments. Nature genetics, 1997. 17(1): p. 15-16.
3. Beharry, S., M. Zhong, and R. S. Molday, N-retinylidene-phosphatidylethanolamine is the preferred retinoid substrate for the photoreceptor-specific ABC transporter ABCA4 (ABCR). The Journal of biological chemistry, 2004. 279(52): p. 53972-53979.
4. Mendell, J. R., et al., Current Clinical Applications of In Vivo Gene Therapy with AAVs. Mol Ther, 2021. 29(2): p. 464-488.
5. Bennett, J., et al., Safety and durability of effect of contralateral-eye administration of AAV2 gene therapy in patients with childhood-onset blindness caused by RPE65 mutations: a follow-on phase 1 trial. Lancet, 2016. 388(10045): p. 661-72.
6. Allikmets, R., et al., Organization of the ABCR gene: analysis of promoter and splice junction sequences. Gene, 1998. 215(1): p. 111-122.
7. Trapani, I., Dual AAV Vectors for Stargardt Disease. Methods in molecular biology (Clifton, N.J.), 2018. 1715: p. 153-175.
8. Miyoshi, H., et al., Stable and efficient gene transfer into the retina using an HIV-based lentiviral vector. Proc Natl Acad Sci USA, 1997. 94(19): p. 10319-23.
9. Han, Z., et al., DNA nanoparticle-mediated ABCA4 delivery rescues Stargardt dystrophy in mice. The Journal of clinical investigation, 2012. 122(9): p. 3221-3226.
10. Komor, A. C., et al., Programmable editing of a target base in genomic DNA without double-stranded DNA cleavage. Nature, 2016. 533(7603): p. 420-424.
11. Gaudelli, N. M., et al., Programmable base editing of A⋅T to G⋅C in genomic DNA without DNA cleavage. Nature, 2017. 551(7681): p. 464-471.
12. Kim, Y. B., et al., Increasing the genome-targeting scope and precision of base editing with engineered Cas9-cytidine deaminase fusions. Nat Biotechnol, 2017. 35(4): p. 371-376.
13. Komor, A. C., et al., Improved base excision repair inhibition and bacteriophage Mu Gam protein yields C:G-to-T:A base editors with higher efficiency and product purity. Sci Adv, 2017. 3(8): p. eaao4774.
14. Rees, H. A., et al., Improving the DNA specificity and applicability of base editing through protein engineering and protein delivery. Nat Commun, 2017. 8: p. 15790.
15. Port, F., et al., Optimized CRISPR/Cas tools for efficient germline and somatic genome engineering in Drosophila. Proc Natl Acad Sci USA, 2014. 111(29): p. E2967-76.
16. Yang, H., H. Wang, and R. Jaenisch, Generating genetically modified mice using CRISPR/Cas-mediated genome engineering. Nat Protoc, 2014. 9(8): p. 1956-68.
17. Yin, H., et al., Genome editing with Cas9 in adult mice corrects a disease mutation and phenotype. Nat Biotechnol, 2014. 32(6): p. 551-3.
18. Carroll, D., Genome editing by targeted chromosomal mutagenesis. Methods Mol Biol, 2015. 1239: p. 1-13.
19. Ran, F. A., et al., In vivo genome editing using Staphylococcus aureus Cas9.

Nature, 2015. 520(7546): p. 186-91.
20. Swiech, L., et al., In vivo interrogation of gene function in the mammalian brain using CRISPR-Cas9. Nat Biotechnol, 2015. 33(1): p. 102-6.
21. Morgens, D. W., et al., Genome-scale measurement of off-target activity using Cas9 toxicity in high-throughput screens. Nat Commun, 2017. 8: p. 15178.
22. Tsai, S. Q., et al., GUIDE-seq enables genome-wide profiling of off-target cleavage by CRISPR-Cas nucleases. Nat Biotechnol, 2015. 33(2): p. 187-197.
23. Cameron, P., et al., Mapping the genomic landscape of CRISPR-Cas9 cleavage. Nat Methods, 2017. 14(6): p. 600-606.
24. Suzuki, K. and J. C. Izpisua Belmonte, In vivo genome editing via the HITI method as a tool for gene therapy. J Hum Genet, 2018. 63(2): p. 157-164.
25. Wang, M., et al., Efficient delivery of genome-editing proteins using bioreducible lipid nanoparticles. Proc Natl Acad Sci USA, 2016. 113(11): p. 2868-73.
26. Kim, K., et al., Highly efficient RNA-guided base editing in mouse embryos. Nat Biotechnol, 2017. 35(5): p. 435-437.
27. Musunuru, K., et al., In vivo CRISPR base editing of PCSK9 durably lowers cholesterol in primates. Nature, 2021. 593(7859): p. 429-434.
28. Levy, J. M., et al., Cytosine and adenine base editing of the brain, liver, retina, heart and skeletal muscle of mice via adeno-associated viruses. Nat Biomed Eng, 2020. 4(1): p. 97-110.
29. Maeder, M. L., et al., Development of a gene-editing approach to restore vision loss in Leber congenital amaurosis type 10. Nat Med, 2019. 25(2): p. 229-233.
30. Nishida, K., et al., Targeted nucleotide editing using hybrid prokaryotic and vertebrate adaptive immune systems. Science, 2016. 353(6305).
31. Rees, H. A. and D. R. Liu, Base editing: precision chemistry on the genome and transcriptome of living cells. Nat Rev Genet, 2018. 19(12): p. 770-788.
32. Choe, D. C. and K. Musunuru, Base editing: a brief review and a practical example. J Biomed Res, 2020. 35(2): p. 107-114.
33. Gu, S., et al., Base Editors: Expanding the Types of DNA Damage Products Harnessed for Genome Editing. Gene Genome Ed, 2021. 1.
34. Chen, Y., et al., Development of Highly Efficient Dual-AAV Split Adenosine Base Editor for In Vivo Gene Therapy. Small Methods, 2020. 4(9): p. 2000309.
35. Winter, J., et al., Targeted exon skipping with AAV-mediated split adenine base editors. Cell Discovery, 2019. 5(1): p. 41.
36. Kantor, A., M. E. McClements, and R. E. MacLaren, CRISPR-Cas9 DNA Base-Editing and Prime-Editing. Int J Mol Sci, 2020. 21(17).
37. Cokol, M., R. Nair, and B. Rost, Finding nuclear localization signals. EMBO Rep, 2000. 1(5): p. 411-5.
38. Freitas, N. and C. Cunha, Mechanisms and signals for the nuclear import of proteins. Curr Genomics, 2009. 10(8): p. 550-7.
39. Muzyczka, N., Use of adeno-associated virus as a general transduction vector for mammalian cells. Curr Top Microbiol Immunol, 1992. 158: p. 97-129.
40. Mingozzi, F. and K. A. High, Therapeutic in vivo gene transfer for genetic disease using AAV: progress and challenges. Nat Rev Genet, 2011. 12(5): p. 341-55.
41. Deyle, D. R. and D. W. Russell, Adeno-associated virus vector integration. Curr Opin Mol Ther, 2009. 11(4): p. 442-7.
42. Asokan, A., D. V. Schaffer, and R. J. Samulski, The AAV vector toolkit: poised at the clinical crossroads. Mol Ther, 2012. 20(4): p. 699-708.
43. Pang, J. J., et al., Comparative analysis of in vivo and in vitro AAV vector transduction in the neonatal mouse retina: effects of serotype and site of administration. Vision Res, 2008. 48(3): p. 377-85.
44. Khani, S. C., et al., AAV-mediated expression targeting of rod and cone photoreceptors with a human rhodopsin kinase promoter. Invest Ophthalmol Vis Sci, 2007. 48(9): p. 3954-61.
45. Allocca, M., et al., Novel adeno-associated virus serotypes efficiently transduce murine photoreceptors. J Virol, 2007. 81(20): p. 11372-80.
46. Tanna, P., et al., Stargardt disease: clinical features, molecular genetics, animal models and therapeutic options. Br J Ophthalmol, 2017. 101(1): p. 25-30.
47. Fujinami, K., et al., ABCA4 gene screening by next-generation sequencing in a British cohort. Investigative ophthalmology & visual science, 2013. 54(10): p. 6662-6674.
48. Riveiro-Alvarez, R., et al., Outcome of ABCA4 disease-associated alleles in autosomal recessive retinal dystrophies: retrospective analysis in 420 Spanish families. Ophthalmology, 2013. 120(11): p. 2332-2337.
49. Valverde, D., et al., Microarray-based mutation analysis of the ABCA4 gene in Spanish patients with Stargardt disease: evidence of a prevalent mutated allele. Mol Vis, 2006. 12: p. 902-8.
50. Zernant, J., et al., Analysis of the ABCA4 gene by next-generation sequencing. Investigative ophthalmology & visual science, 2011. 52(11): p. 8479-8487.
51. Consugar, M. B., et al., Panel-based genetic diagnostic testing for inherited eye diseases is highly accurate and reproducible, and more sensitive for variant detection, than exome sequencing. Genet Med, 2015. 17(4): p. 253-261.
52. Leroy, B. P., et al., Spectrum of mutations in USH2A in British patients with Usher syndrome type II. Exp Eye Res, 2001. 72(5): p. 503-9.
53. Yan, D., et al., Mutation analysis in the long isoform of USH2A in American patients with Usher Syndrome type II. J Hum Genet, 2009. 54(12): p. 732-8.
54. Valverde, D., et al., Microarray-based mutation analysis of the ABCA4 gene in Spanish patients with Stargardt disease: evidence of a prevalent mutated allele. Molecular vision, 2006. 12: p. 902-908.
55. Hu, J. H., et al., Evolved Cas9 variants with broad PAM compatibility and high DNA specificity. Nature, 2018. 556(7699): p. 57-63.
56. Clement, K., et al., CRISPResso2 provides accurate and rapid genome editing sequence analysis. Nat Biotechnol, 2019. 37(3): p. 224-226.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A composition comprising a single guide RNA (sgRNA) comprising a Protospacer Sequence comprising one of SEQ ID NOs:6 to 37, and optionally a corresponding base editor (BE), optionally an adenine base editor (ABE) or cytosine base editor (CBE).

2. The composition of claim 1, comprising ribonucleoprotein complexes comprising the sgRNA/BE.

3. The composition of claim 1, comprising nucleic acids encoding the sgRNA/BE, or the sgRNA and nucleic acids encoding the BE, respectively.

4. The composition of claim 3, wherein the nucleic acids comprise mRNA or cDNA encoding the BE proteins, optionally wherein the nucleic acids are naked or in an expression vector comprising a regulatory sequence such as a promoter that drives expression of the protein.

5. The composition of claim 4, wherein the nucleic acids comprising mRNA or cDNA encoding the BE proteins further comprise a nuclear localization signal, optionally a C-terminal nuclear localization signal and/or an N-terminal nuclear localization signal; and/or wherein the sequences encoding the BE comprise a polyadenylation signal.

6. The composition of claim 4, wherein the expression vectors are viral delivery vectors, optionally an AAV, preferably wherein the AAV further comprises a first and a second inverted terminal repeat sequence (ITR).

7. The composition of claim 6, wherein the viral delivery vector comprises a promoter that drives expression of the BE, preferably a CMV, EFS, or hGRK1 promoter.

8. (canceled)

9. (canceled)

10. (canceled)

11. (canceled)

12. (canceled)

13. A method of altering a genome of a cell, comprising contacting the cell with the composition of claim 1.

14. The method of claim 13, wherein the cell is from a subject who has Stargardt disease.

15. The method of claim 13, wherein the cell is a retinal cell or a photoreceptor cell.

16. The method of claim 15, wherein the photoreceptor cell is a cone photoreceptor cell or a cone cell, a rod photoreceptor cell or a rod cell or a macular cone photoreceptor cell.

17. A method of altering a cell, comprising contacting the cell with a recombinant viral particle comprising:
a nucleotide sequence encoding an sgRNA comprising a Protospacer Sequence comprising one of SEQ ID NOs:6 to 37, and a corresponding base editor (BE), optionally an adenine base editor (ABE) or cytosine base editor (CBE), or
wherein said viral particle is capable of delivery to a non-dividing cell, and wherein said contacting results in correction of a pathogenic mutation in an ABCA4 allele in the cell.

18. The method of claim 17, wherein the viral particle is an adeno-associated virus (AAV) viral particle.

19. A method of treating a subject who has a condition associated with a mutation in an ABCA4 allele, the method comprising administering to the subject a therapeutically effective amount of the composition of claim 1.

20. The method of claim 19, wherein the subject has Stargardt disease.

21. The method of claim 19, wherein the cell is a retinal cell or a photoreceptor cell.

22. The method of claim 21, wherein the composition is delivered to a retina of a subject by injection, such as by subretinal injection.

23. The method of claim 22, wherein the photoreceptor cell is a cone photoreceptor cell or a cone cell, a rod photoreceptor cell or a rod cell or a macular cone photoreceptor cell.


SEQUENCE LISTING (94 sequences)

SEQ ID NO: 1 - protein, 7 aa, synthetic construct
PKKKRKV

SEQ ID NO: 2 - protein, 7 aa, synthetic construct
PKKKRRV

SEQ ID NO: 3 - protein, 16 aa, synthetic construct
KRPAATKKAGQAKKKK

SEQ ID NO: 4 - DNA, 60 bp, synthetic construct
  source 1..60: mol_type = other DNA
tagcaataaaggatcgtttattttcattggaagcgtgtgttggttttttgatcaggcgcg

SEQ ID NO: 5 - DNA, 264 bp, synthetic construct
  source 1..264: mol_type = other DNA
aaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 6 - DNA, 20 bp, synthetic construct
  source 1..20: mol_type = other DNA
tgtcgaagttcgccctggag

SEQ ID NO: 7 - DNA, 20 bp, synthetic construct
  source 1..20: mol_type = other DNA
cgaagttcgccctggagagg

SEQ ID NO: 8 - DNA, 20 bp, synthetic construct
  source 1..20: mol_type = other DNA
tgtgtcgaagttcgccctgg

SEQ ID NO: 9 - DNA, 20 bp, synthetic construct
  source 1..20: mol_type = other DNA
tcgaagttcgccctggagag

SEQ ID NO: 10 - DNA, 20 bp, synthetic construct
  source 1..20: mol_type = other DNA
ctccagctggacctcctcct

SEQ ID NO: 11 - DNA, 20 bp, synthetic construct
  source 1..20: mol_type = other DNA
tccagctggacctcctcctg

SEQ ID NO: 12 - DNA, 20 bp, synthetic construct
  source 1..20: mol_type = other DNA
ccagctggacctcctcctgg

SEQ ID NO: 13 - DNA, 20 bp, synthetic construct
  source 1..20: mol_type = other DNA
ctctccactggaggaaaaca

SEQ ID NO: 14 - DNA, 20 bp, synthetic construct
  source 1..20: mol_type = other DNA
gtacatccatgccacaccct

SEQ ID NO: 15 - DNA, 20 bp, synthetic construct
  source 1..20: mol_type = other DNA
ggtacatccatgccacaccc

SEQ ID NO: 16 - DNA, 20 bp, synthetic construct
  source 1..20: mol_type = other DNA
acatccatgccacaccctgg

SEQ ID NO: 17 - DNA, 20 bp, synthetic construct
  source 1..20: mol_type = other DNA
actgctacccatatatccag

SEQ ID NO: 18 - DNA, 20 bp, synthetic construct
  source 1..20: mol_type = other DNA
tactgctacccatatatcca

SEQ ID NO: 19 - DNA, 20 bp, synthetic construct
  source 1..20: mol_type = other DNA
gctacccatatatccagggg

SEQ ID NO: 20 - DNA, 20 bp, synthetic construct
  source 1..20: mol_type = other DNA
gtactgctacccatatatcc

SEQ ID NO: 21 - DNA, 20 bp, synthetic construct
  source 1..20: mol_type = other DNA
acagcagttcaggatgatca

SEQ ID NO: 22 - DNA, 20 bp, synthetic construct
  source 1..20: mol_type = other DNA
agggaaacagcagttcagga

SEQ ID NO: 23 - DNA, 20 bp, synthetic construct
  source 1..20: mol_type = other DNA
ctgagcaatacttcaggagc

SEQ ID NO: 24 - DNA, 20 bp, synthetic construct
  source 1..20: mol_type = other DNA
tgagcaatacttcaggagca

SEQ ID NO: 25 - DNA, 20 bp, synthetic construct
  source 1..20: mol_type = other DNA
acctgagcaatacttcagga

SEQ ID NO: 26 - DNA, 20 bp, synthetic construct
  source 1..20: mol_type = other DNA
agcagggtatgtaaacagac

SEQ ID NO: 27 - DNA, 20 bp, synthetic construct
  source 1..20: mol_type = other DNA
tgtgagcagggtatgtaaac

SEQ ID NO: 28 - DNA, 20 bp, synthetic construct
  source 1..20: mol_type = other DNA
ccagcaccacttcttcctct

SEQ ID NO: 29 - DNA, 20 bp, synthetic construct
  source 1..20: mol_type = other DNA
tacttccctgtttcagacgc

SEQ ID NO: 30 - DNA, 20 bp, synthetic construct
  source 1..20: mol_type = other DNA
aggactcctgtggctgcctc

SEQ ID NO: 31 - DNA, 20 bp, synthetic construct
  source 1..20: mol_type = other DNA
atgctgaggactcctgtggc

SEQ ID NO: 32 - DNA, 20 bp, synthetic construct
  source 1..20: mol_type = other DNA
gtagccaggagcacgatctg

SEQ ID NO: 33 - DNA, 20 bp, synthetic construct
  source 1..20: mol_type = other DNA
tagccaggagcacgatctgt

SEQ ID NO: 34 - DNA, 20 bp, synthetic construct
  source 1..20: mol_type = other DNA
agccaggagcacgatctgtg

SEQ ID NO: 35 - DNA, 20 bp, synthetic construct
  source 1..20: mol_type = other DNA
cctgtgaacagctcatcaat

SEQ ID NO: 36 - DNA, 20 bp, synthetic construct
  source 1..20: mol_type = other DNA
tgtgaacagctcatcaattg

SEQ ID NO: 37 - DNA, 20 bp, synthetic construct
  source 1..20: mol_type = other DNA
caggtacagcaccagggaga

SEQ ID NO: 38 - DNA, 51 bp, synthetic construct
  source 1..51: mol_type = other DNA
ccagcagtggacaggctgtgtgtcgaagttcgccctggagaggtgggtact

SEQ ID NO: 39 - DNA, 51 bp, synthetic construct
  source 1..51: mol_type = other DNA
ctgaaaggaaagtcccaggaggaggtccagctggagatggaagccatgttg

SEQ ID NO: 40 - DNA, 51 bp, synthetic construct
  source 1..51: mol_type = other DNA
cagctcacccaacgtgccctctctccactggaggaaaacatgttctgggcc

SEQ ID NO: 41 - DNA, 77 bp, synthetic construct
  source 1..77: mol_type = other DNA
ttcctcctgaccctgctggtccagcaccacttcttcctctcccaatggtacatccatgccacaccctgggccagtgg

SEQ ID NO: 42 - DNA, 51 bp, synthetic construct
  source 1..51: mol_type = other DNA
gacccttcacccctggatatatgggtagcagtacaccttcttcaggtgcgc

SEQ ID NO: 43 - DNA, 51 bp, synthetic construct
  source 1..51: mol_type = other DNA
ctttagtttcatgatcatcctgaactgctgtttccctatcttcatggtgct

SEQ ID NO: 44 - DNA, 51 bp, synthetic construct
  source 1..51: mol_type = other DNA
aatctgggatctgctcctgaagtattgctcaggtaacagctgctgctcagt

SEQ ID NO: 45 - DNA, 51 bp, synthetic construct
  source 1..51: mol_type = other DNA
ccttggccggatcatgaatgtgagcagggtatgtaaacagactggagattt

SEQ ID NO: 46 - skipped

SEQ ID NO: 47 - DNA, 51 bp, synthetic construct
  source 1..51: mol_type = other DNA
gccccgtttccaacagtcctacttccctgtttcagacgctgctcaggttca

SEQ ID NO: 48 - DNA, 51 bp, synthetic construct
  source 1..51: mol_type = other DNA
gtttgctttgctgtcaatgctgaggactcctgtggctgcctccactcaaaa

SEQ ID NO: 49 - DNA, 51 bp, synthetic construct
  source 1..51: mol_type = other DNA
tctattctcccacagatcgtgctcctggctacctttgtgtttttggctctg

SEQ ID NO: 50 - DNA, 51 bp, synthetic construct
  source 1..51: mol_type = other DNA
tcagtttgatgcaattgatgagctgttcacaggacgagaacatctttacct

SEQ ID NO: 51 - DNA, 51 bp, synthetic construct
  source 1..51: mol_type = other DNA
gtcaacccttcaccatcctgcaggtacagcaccagggagaagctcaccatg

SEQ ID NO: 52 - DNA, 13 bp, synthetic construct
  source 1..13: mol_type = other DNA
actgctacccata

SEQ ID NO: 53 - DNA, 13 bp, synthetic construct
  source 1..13: mol_type = other DNA
aaacagcagttca

SEQ ID NO: 54 - DNA, 13 bp, synthetic construct
  source 1..13: mol_type = other DNA
agcagggtatgta

SEQ ID NO: 55 - DNA, 19 bp, synthetic construct
  source 1..19: mol_type = other DNA
ctgttcctgtacggcatgg

SEQ ID NO: 56 - DNA, 114 bp, synthetic construct
  source 1..114: mol_type = other DNA
gccctgttcctgtacggcatggccagcagtggacaggctgtgtgtcgaagttcgccctggagaggtgggtactctgaaaggaaagtcccaggaggaggtccagctggagatgga

SEQ ID NO: 57 - DNA, 114 bp, synthetic construct
  source 1..114: mol_type = other DNA
cgggacaaggacatgccgtaccggtcgtcacctgtccgacacacagcttcaagcgggacctctccacccatgagactttcctttcagggtcctcctccaggtcgacctctacct

SEQ ID NO: 58 - protein, 38 aa, synthetic construct
AIFIYGMASSGOAVCRSSPWRGGYSFRKVPGGGPAGDG

SEQ ID NO: 59 - DNA, 111 bp, synthetic construct
  source 1..111: mol_type = other DNA
ctgttcctgtacggcatggccagcagtggacaggctgtgtgtcgaagttcgccctggagaggtgggtactctgaaaggaaagtcccaggaggaggtccagctggagatgga

SEQ ID NO: 60 - DNA, 111 bp, synthetic construct
  source 1..111: mol_type = other DNA
ctgttcctgtacggcatggccagcagtggacaggctgtgtgtcggggttcgccctggagaggtgggtactctgaaaggaaagtcccaggaggaggtccagctggagatgga

SEQ ID NO: 61 - DNA, 115 bp, synthetic construct
  source 1..115: mol_type = other DNA
cctttttcctcctgaccctgctggtccagcaccacttcttcctctcccaatggtacatccatgccacaccctgggccagtgggacccttcacccctggatatatgggtagcagta

SEQ ID NO: 62 - DNA, 115 bp, synthetic construct
  source 1..115: mol_type = other DNA
ggaaaaaggaggactgggacgaccaggtcgtggtgaagaaggagagggttaccatgtaggtacggtgtgggacccggtcaccctgggaagtggggacctatatacccatcgtcat

SEQ ID NO: 63 - protein, 37 aa, synthetic construct
PFSSPCWSSTTSSSPNGTSMPHPGPVGPFTPGYMGSS

SEQ ID NO: 64 - protein, 38 aa, synthetic construct
LFPPDPAGPAPIIPIPMVHPCHTIGQWDPSPIDIWVAV

SEQ ID NO: 65 - DNA, 115 bp, synthetic construct
  source 1..115: mol_type = other DNA
cctttttcctcctgaccctgctggtccagcaccacttcttcctctcccaatggtgcgtccgtgccacaccctgggccagtgggacccttcacccctggatatatgggtagcagta

SEQ ID NO: 66 - DNA, 115 bp, synthetic construct
  source 1..115: mol_type = other DNA
cctttttcctcctgaccctgctggtccagcaccacttcttcctctcccaatggtacgtccatgccacaccctgggccagtgggacccttcacccctggatatatgggtagcagta

SEQ ID NO: 67 - DNA, 115 bp, synthetic construct
  source 1..115: mol_type = other DNA
cctttttcctcctgaccctgctggtccagcaccacttcttcctctcccaatggtgcgtccatgccacaccctgggccagtgggacccttcacccctggatatatgggtagcagta

SEQ ID NO: 68 - DNA, 115 bp, synthetic construct
  source 1..115: mol_type = other DNA
cctttttcctcctgaccctgctggtccagcaccacttcttcctctcccaatggtacgtccgtgccacaccctgggccagtgggacccttcacccctggatatatgggtagcagta

SEQ ID NO: 69 - DNA, 40 bp, synthetic construct
  source 1..40: mol_type = other DNA
agtgggacccttcacccctggatatatgggtagcagtaca

SEQ ID NO: 70 - DNA, 40 bp, synthetic construct
  source 1..40: mol_type = other DNA
agtgggacccttcacccctggatatatgggcagcagtaca

SEQ ID NO: 71 - DNA, 40 bp, synthetic construct
  source 1..40: mol_type = other DNA
agtgggacccttcacccctggatatacgggcagcagtaca

SEQ ID NO: 72 - DNA, 40 bp, synthetic construct
  source 1..40: mol_type = other DNA
agtgggacccttcacccctggatacatgggtagcagtaca

SEQ ID NO: 73 - DNA, 40 bp, synthetic construct
  source 1..40: mol_type = other DNA
agtgggacccttcacccctggatatacgggtagcagtaca

SEQ ID NO: 74 - DNA, 40 bp, synthetic construct
  source 1..40: mol_type = other DNA
agtgggacccttcacccctggatacatgggcagcagtaca

SEQ ID NO: 75 - DNA, 40 bp, synthetic construct
  source 1..40: mol_type = other DNA
gtgcgcctttagtttcatgatcatcctgaactgctgtttc

SEQ ID NO: 76 - DNA, 40 bp, synthetic construct
  source 1..40: mol_type = other DNA
gtgcgcctttagtttcatgatcatcctgaaccgctgtttc

SEQ ID NO: 77 - DNA, 40 bp, synthetic construct
  source 1..40: mol_type = other DNA
gtgcgcctttagtttcatgatcatcctgaactgccgtttc

SEQ ID NO: 78 - DNA, 40 bp, synthetic construct
  source 1..40: mol_type = other DNA
gtgcgcctttagtttcatgatcatcctgaaccgccgtttc

SEQ ID NO: 79 - DNA, 40 bp, synthetic construct
  source 1..40: mol_type = other DNA
caagcaatctgggatctgctcctgaagtattgctcaggta

SEQ ID NO: 80 - DNA, 40 bp, synthetic construct
  source 1..40: mol_type = other DNA
caagcaatctgggatctgctcctgaagtatcgctcaggta

SEQ ID NO: 81 - DNA, 40 bp, synthetic construct
  source 1..40: mol_type = other DNA
caagcaatctgggatctgctcctgaagtaccgctcaggta

SEQ ID NO: 82 - DNA, 40 bp, synthetic construct
  source 1..40: mol_type = other DNA
caagcaatctgggatctgctcctgaagcatcgctcaggta

SEQ ID NO: 83 - DNA, 40 bp, synthetic construct
  source 1..40: mol_type = other DNA
caagcaatctgggatctgctcctgaagcattgctcaggta

SEQ ID NO: 84 - DNA, 40 bp, synthetic construct
  source 1..40: mol_type = other DNA
caagcaatctgggatctgctcctgaagtactgctcaggta

SEQ ID NO: 85 - DNA, 40 bp, synthetic construct
  source 1..40: mol_type = other DNA
caagcaatctgggatctgctcctgaagtatcgcccaggta

SEQ ID NO: 86 - DNA, 40 bp, synthetic construct
  source 1..40: mol_type = other DNA
caagcaatctgggatctgctcctgaagtattgcccaggta

SEQ ID NO: 87 - DNA, 40 bp, synthetic construct
  source 1..40: mol_type = other DNA
caagcaatctgggatctgctcctgaagcaccgctcaggta

SEQ ID NO: 88 - DNA, 40 bp, synthetic construct
  source 1..40: mol_type = other DNA
caagcaatctgggatctgctcctgaagtaccgcccaggta

SEQ ID NO: 89 - DNA, 115 bp, synthetic construct
  source 1..115: mol_type = other DNA
aacagctgctgctcagtccttggccggatcatgaatgtgagcagggtatgtaaacagactggagatttgccccgtttccaacagtcctacttccctgtttcagacgctgctcagg

SEQ ID NO: 90 - DNA, 115 bp, synthetic construct
  source 1..115: mol_type = other DNA
ttgtcgacgacgagtcaggaaccggcctagtacttacactcgtcccatacatttgtctgacctctaaacggggcaaaggttgtcaggatgaagggacaaagtctgcgacgagtcc

SEQ ID NO: 91 - protein, 39 aa, synthetic construct
DQLLLSPWPDHECEOGMMTDWRFAPFPTVIIPCFBBCSG

SEQ ID NO: 92 - DNA, 115 bp, synthetic construct
  source 1..115: mol_type = other DNA
aacagctgctgctcagtccttggccggatcatgaatgtgagcggggtgtgtgaacagactggagatttgccccgtttccaacagtcctacttccctgtttcagacgctgctcagg

SEQ ID NO: 93 - DNA, 115 bp, synthetic construct
  source 1..115: mol_type = other DNA
aacagctgctgctcagtccttggccggatcatgaatgtgagcggggtgtgtaaacagactggagatttgccccgtttccaacagtcctacttccctgtttcagacgctgctcagg

SEQ ID NO: 94 - DNA, 115 bp, synthetic construct
  source 1..115: mol_type = other DNA
aacagctgctgctcagtccttggccggatcatgaatgtgagcggggtatgtaaacagactggagatttgccccgtttccaacagtcctacttccctgtttcagacgctgctcagg